(19) United States
(12) Patent Application Publication
(10) Pub. No.: US 2021/0212619 A1
(43) Pub. Date: Jul. 15, 2021

(54) Cognitive Readiness Determination and Control System and Method

(71) Applicant: Paxmentys, LLC, Naperville, IL (US)

(72) Inventors: Irene Crockett, Naperville, IL (US); Mark Crockett, Naperville, IL (US)

(21) Appl. No.: 17/146,448

(22) Filed: Jan. 11, 2021

Related U.S. Application Data
(60) Provisional application No. 62/960,449, filed on Jan. 13, 2020.

(51) Int. Cl. A61B 5/16; H04L 29/06

(52) U.S. Cl. CPC A61B 5/162; H04L 63/0815; H04L 63/0861; A61B 5/165; H04L 63/0838

(57) ABSTRACT

A cognitive readiness determination and control system and method is disclosed. The system is configured to send to or prompt a cognitive readiness test on the client electronic device of the user in response to a request for access to a computing resource. The cognitive readiness test comprises the presentation of a stimulus on the client electronic device. The system is configured to control access to the computing resource or notify another based on a reaction time of the user to the stimulus.

This application claims the benefit of U.S. Patent Application No. 62/960,449, filed Jan. 13, 2020, which is herein incorporated by reference in its entirety.

### FIELD OF THE INVENTION

This invention relates in general to cognitive readiness determination and control systems and methods.

### BACKGROUND OF THE INVENTION

Access to software or electronic devices may be restricted based on authentication credentials, such as a user name and password. However, at least one of the present inventors recognized the need for a system for determining a user's cognitive readiness to use a sought computing resource at the time of access or use. Further, at least one of present inventors recognized the need for a system that denies access to the sought computing resource and/or notifies another when a user lacks cognitive readiness.

A person can be cognitively impaired for a variety of reasons, including alcohol or drug use, sleep deprivation, drug use, age, or illness, depression, among other biologic and environmental factors. When a person endeavors to perform at work, in school, or in other contexts, and is cognitively impaired, he/she is prone to errors in judgement and performance, which can lead to injury. Examples of impairment include a physician who is working while sleep deprived and injures a patient during surgery or an engineer who is working on a design while acutely marijuana intoxicated leading to errors in judgement and lack of productivity.

Although there are methods to measure acute alcohol intoxication, such as by measuring breath alcohol or blood alcohol levels, many other drugs or impairment factors have not been easy to measure. For example, tetrahydrocannabinol (THC), which is the of the principle psychoactive constituent of cannabis or marijuana, is known to have higher concentration in the brain than in blood, and persists long after acute intoxication. Therefore measuring blood levels or urine levels of THC is therefore not useful in determining between recent or remote use of the drug or whether a person is acutely intoxicated.

At least one of the present inventors recognized the need for a cognitive readiness measurement system and method that is convenient to implement on small and large scales that can be used for notification and/or control of access. At least one of the present inventors recognized the need for a cognitive readiness system and method that can be used in high reliability organizations or in high risk or high stakes environments. At least one of the present inventors recognized the need for a cognitive readiness control in single sign-on environments.

At least one of the present inventors recognized the need for a new system for authenticating a user and determining their cognitive readiness or impairment. At least one of the present inventors recognized the need for controlling access to computer resources based on a user's cognitive readiness or impairment.

At least one of the present inventors recognized the need for a system of cognitive readiness testing that is integrated within a sign-on process to provide screening at the beginning of a work experience or work day for increasing organizational and personal safety. At least one of the present inventors recognized the need for a system of cognitive readiness testing that is integrated within a software that the user already uses for other purposes to provide no or minimal disruption during successful cognitive screening of the user.

### SUMMARY OF THE INVENTION

A method of controlling access to a computer system is disclosed. One embodiment of the method involves the following. A request for access to a target computing resource is received from a client electronic device. One or more user authentication credentials of a user are received at an identity provider from a client electronic device. A cognitive readiness test is sent to the client electronic device of the user. Access to the target computing resource is granted or denied based on a reaction time of the user to a stimulus of the cognitive readiness test.

In some embodiments, access to the target computing resource is granted if a reaction time of the user to the cognitive readiness test is within a predefined range of acceptable reaction times. In some embodiments, one or more user authentication credentials of a user are received at an identity provider from a client electronic device. The identity provider is a single sign-on authority and the single sign-on authority determines whether the authentication credentials are valid to establish user identity. Access to the target computer system is granted if the authentication credentials are valid and a reaction time of the user to the cognitive readiness test is within a predefined range of acceptable reaction times, and therefore user identity and readiness are established.

In some embodiments, the cognitive readiness test is sent by a cognitive authority and the cognitive authority determines whether the reaction time of the user to the cognitive readiness test is within a predefined range of acceptable reaction times. In some embodiments, the single sign-on authority includes the cognitive authority.

In some embodiments, the predefined range of acceptable reaction times is a user personalized predefined range of acceptable reaction times determined based on one or more previous reaction times of one or more previous cognitive readiness tests taken by the user. In some embodiments, the predefined range of acceptable reaction times is a user personalized predefined range of acceptable reaction times determined based on one or more pieces of demographic information available to the system.

In some embodiments, the cognitive readiness test is presented on a graphical user interface of the client electronic device. The user's reaction time to the stimulus presented in the cognitive readiness test is determined by measuring a time between the presentation of the stimulus on the graphical user interface and the receipt of a predefined user input on the client electronic device.

In some embodiments, the cognitive test is presented via one or more illuminating elements on the client electronic device. The illuminating elements provide a stimulus by illumination.

The user's reaction time to a stimulus is determined by measuring a time between the presentation of the stimulus and the receipt of a predefined user input on the client electronic device.

A computing resource access control system is disclosed having a processor and a memory. The memory has a plurality of program instructions stored thereon that are executable by the processor to perform operations including sending a cognitive readiness test to a client electronic device of a user in response to a request for access to a computing resource, where the cognitive readiness test comprises the presentation of a stimulus on the client electronic device, and, controlling access to the computing resource based on a reaction time of the user to the stimulus of the cognitive readiness test.

In some embodiments, the system may log or notify another rather than or in addition to granting or denying access to the target computing resource.

In some embodiments, the access control system has a target computer and a sign-on authority. The target computer has a request receiving function configured to receive a request for access to the target computer from a client electronic device. The sign-on authority has a request authentication credentials function configured request authentication credentials from a user at the client electronic device. The sign-on authority has an authentication credentials receiving function configured to receive from the user one or more authentication credentials. In some embodiments, the sign-on authority has a send cognitive readiness test function configured to send a cognitive readiness test to the client electronic device of the user in connection with requesting authentication credentials from a user. The sign-on authority has a grant function configured to grant access to the target computer if the authentication credentials are valid and a reaction time of the user to the cognitive readiness test is within a predefined range of acceptable reaction times. In some embodiments, the sign-on authority is a single sign-on authority.

Numerous other advantages and features of the present invention will become readily apparent from the following detailed description of the invention and the embodiments thereof, from the claims, and from the accompanying drawings.

### BRIEF DESCRIPTION OF THE DRAWINGS

FIG. 1 is a flow diagram of a first embodiment access control function of a cognitive readiness determination system of the invention.

FIG. 2 is a block diagram of a first embodiment of a target computing resource and a cognitive authority of the cognitive readiness determination system.

FIG. 3 is a block diagram of a second embodiment of a target computing resource and a cognitive authority of the cognitive readiness determination system.

FIG. 4A is a block diagram of a third embodiment of a target computing resource and a cognitive authority of the cognitive readiness determination system.

FIG. 4B is a block diagram of a fourth embodiment of a target computing resource and a cognitive authority of the cognitive readiness determination system.

FIG. 5 is a block diagram of an example operating environment for the cognitive readiness determination system.

FIG. 6 is a flow diagram of one embodiment of a cognitive test result evaluation function of the cognitive readiness determination system.

FIG. 7 is a flow diagram of a login or access sequence where the cognitive readiness determination system can be used.

FIG. 8 is a flow diagram showing a first embodiment of communications flows in a single sign-on environment using the cognitive readiness determination system.

FIG. 9A is a simplified flow diagram showing the first embodiment of communications flows in a single sign-on environment using the cognitive readiness determination system where the cognitive authority is integrated into the single sign-on authority.

FIG. 9B is a block diagram of a resource/system target, a cognitive authority, and a single sign-on authority of an embodiment of the cognitive readiness determination system.

FIG. 9C is a block diagram of example portions of identity stores of an embodiment of the cognitive readiness determination system.

FIG. 10A is a flow diagram of a second embodiment access control function of the cognitive readiness determination system of the invention.

FIG. 10B is exemplary user input interface.

FIG. 11 is a user record of an embodiment of the cognitive readiness determination system.

FIG. 12 is a first embodiment cognitive readiness test user interface of the cognitive readiness determination system.

FIG. 13 is the first embodiment cognitive readiness test user interface of FIG. 12 in a second stage of use.

FIG. 14 is a cognitive test pass user interface of the cognitive readiness determination system.

FIG. 15 is a cognitive test fail user interface of the cognitive readiness determination system.

FIG. 16 is a second embodiment cognitive readiness test user interface of the cognitive readiness determination system.

FIG. 17 is a third embodiment cognitive readiness test user interface of the cognitive readiness determination system in a first stage of use.

FIG. 18 is the cognitive readiness test user interface of FIG. 17 in a second stage of use.

FIG. 19 is the cognitive readiness test user interface of FIG. 17 in a third stage of use.

FIG. 20 is a fourth embodiment cognitive readiness test user interface of the cognitive readiness determination system in a first stage of use.

FIG. 21 is the fourth embodiment cognitive readiness test user interface of FIG. 20 in a second stage of use.

FIG. 22 is block diagram of a first embodiment housing of an embodiment of the user client.

FIG. 23 is block diagram of a second embodiment housing of an embodiment of the user client.

FIG. 24 is a block diagram of a third embodiment housing of an embodiment of the user client.

FIG. 25 is a block diagram of an example server computer architecture usable with the cognitive readiness determination system.

FIG. 26 is a block diagram of an example user client architecture usable with the cognitive readiness determination system.

### DETAILED DESCRIPTION

The following description is presented to enable any person skilled in the art to make and use the invention. For the purposes of explanation, specific nomenclature is set forth to provide a plural understanding of the present invention. While this invention is susceptible of embodiment in many different forms, this description describes and the drawings show specific embodiments of the invention with the understanding that the present disclosure is to be considered an exemplification of the principles of the invention and is not intended to limit the invention to the specific embodiments illustrated.

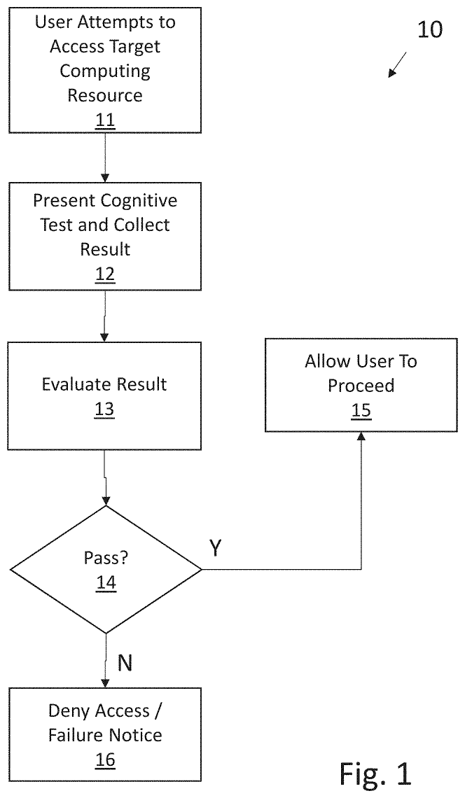

FIG. 1 shows a flow diagram an access control function 10 of the cognitive readiness determination system of the invention. In some embodiments, the cognitive readiness determination system comprises a cognitive authority 30 and a target computing resource 32. In some embodiments, the cognitive readiness determination system comprises the cognitive authority 30, a user client 31, the target computing resource 32, and a single sign-on (SSO) authority 34.

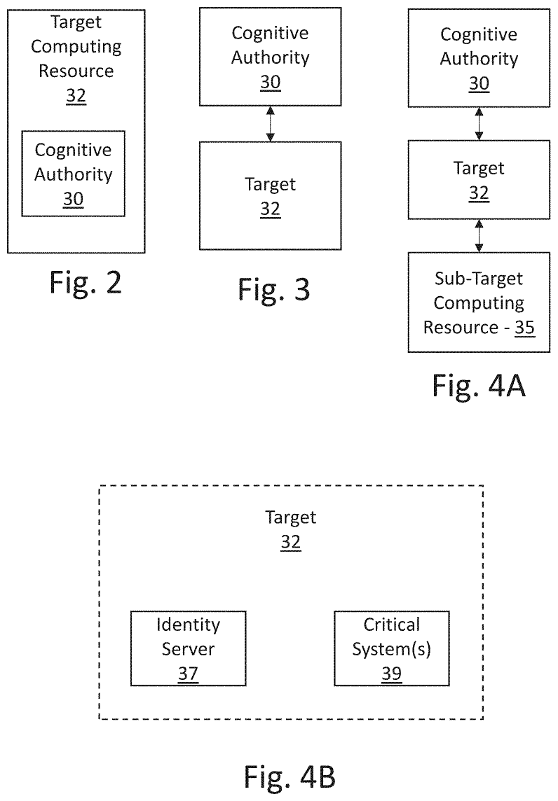

In some embodiments, the target 32 comprises the cognitive authority 30, as shown in FIG. 2. In some embodiments, the cognitive authority 30 is connected or otherwise in communication with the target 32, as shown in FIG. 3. In some embodiments, the cognitive authority is connected or otherwise in communication with the target 32, which is connected to one or more target sub-system(s) or resource(s) 35, which is shown in FIG. 4A.

In some embodiments, the target computing resource 32 may comprise one or more of a computing device(s), a system resource(s), a program(s), a software(s) or application(s), and/or a webpage(s) or website(s), and/or a portion(s) thereof. In some embodiments, computing a device(s) comprise a computer(s) and/or a computer system(s). In some embodiments, a system resource may comprise any physical or virtual component of a computer or computer system, including but not limited to a drive, a file, a folder, a directory, a network location, a data storage location, a data, or a database.

In some embodiments, the cognitive readiness determination system is configured to control access to, or facilitate the control of access to, a computing resource based a user's the cognitive readiness. In some embodiments, the cognitive readiness determination system is configured to record cognitive readiness information of a first user and/or notify second person or user based on first user's the cognitive readiness.

In some embodiments, when a user is attempting to access the target computing resource 32, the user is attempting to access a sub-target computing resource of the target computing resource or accessible through or in connection with the target computing resource. In some embodiments, the target computing resource 32 comprises an identity server 37 and/or one or more critical computer system(s) 39, as shown in FIG. 4B.

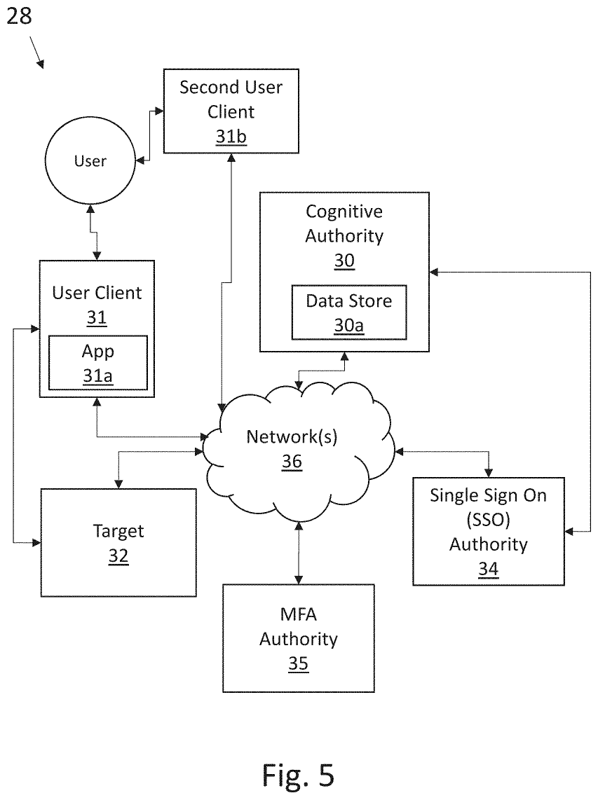

FIG. 5 shows an operating environment 28 where the cognitive authority 30 is connected, directly or through one or more networks 35 and to a single sign-on (SSO) authority 34. The SSO authority is connected, directly or through one or more networks 36, to the target 32. The user client is in communication with the target computing resource 32. The user client is also in communication with at least the SSO authority. The user client may be connected directly to the target 32 or through the one or more networks 36 to the target 32. The user client may be or comprise a client electronic device.

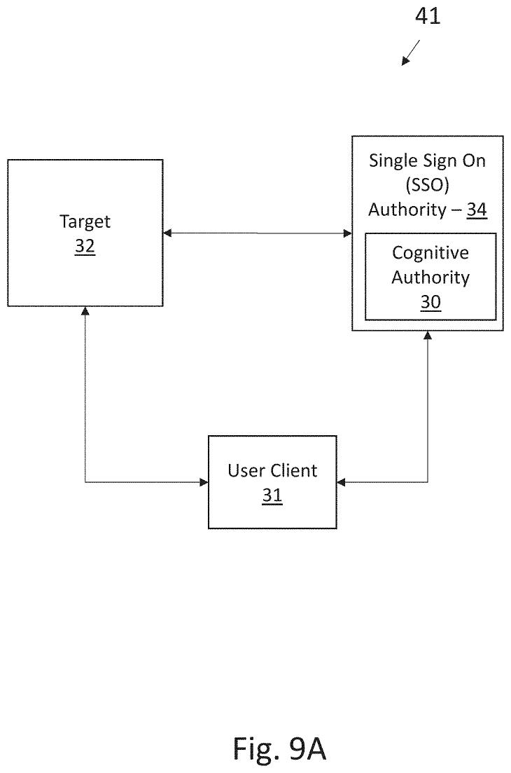

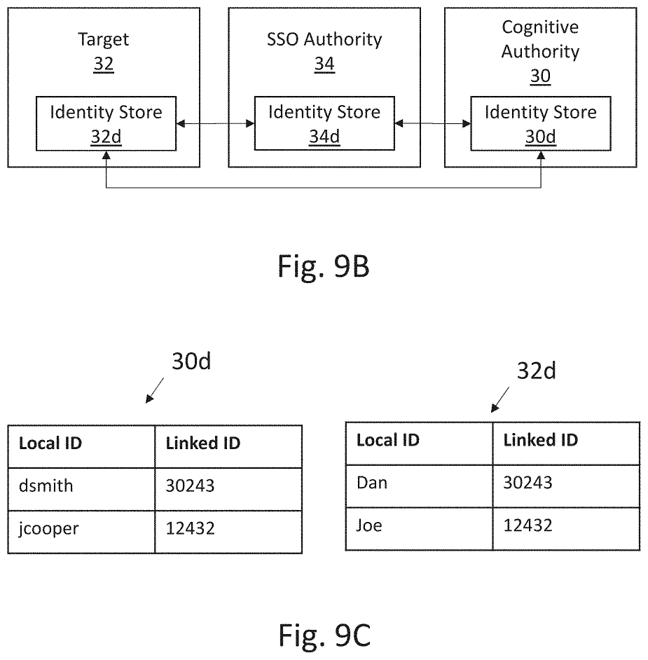

In some embodiments, the SSO authority comprises the cognitive authority. In such case, the cognitive authority may be integrated into the SSO authority, as shown in FIG. 9A.

At step 11 of FIG. 1, a user requests or attempts to access a target 32. In some embodiments, the user may request or attempt to access to the target computing resource 32 by, without limitation, navigating to a login screen, navigating to a webpage with a uniform resource locator (URL), selecting user interface element, or/and entering a command, corresponding to the target computing resource.

At step 12 the cognitive authority 30 sends or presents a cognitive test to the user client of the user and receives the results of the cognitive test. The cognitive test is configured to measure or assess one or more cognitive markers indicative of a user's cognitive readiness. In some embodiments, the cognitive markers, comprise a reaction time to a stimulus and/or the ability to recall and repeat a sequence. In some embodiments and applications, the cognitive test may be referred to as a cognitive game. In some embodiments, the cognitive test is already resident on the user client and at step 12 the cognitive authority sends instructions for the user client to begin the cognitive test or to prompt the user to begin the cognitive test.

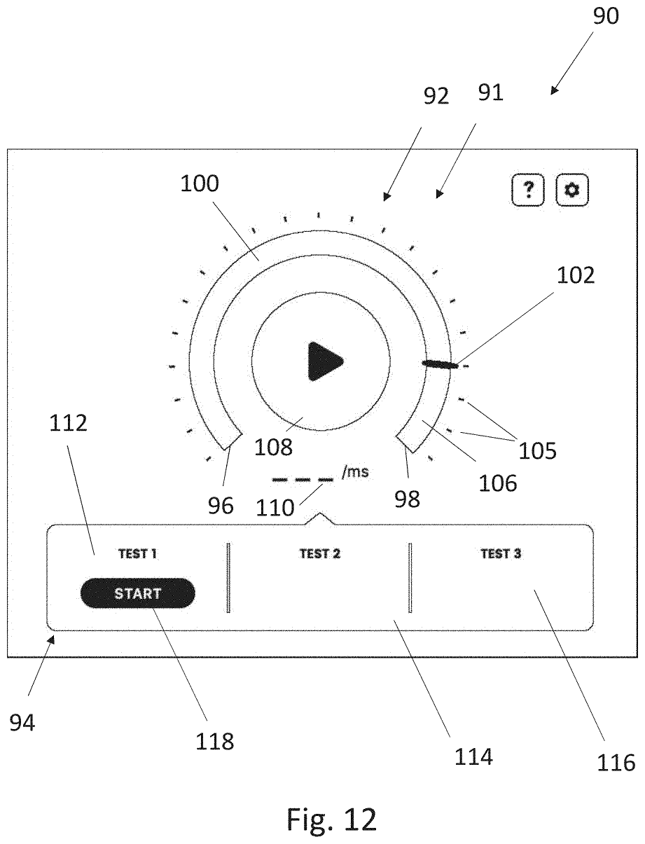

FIGS. 12-13, 16, and 18-21 show exemplary cognitive tests, which will be explained further below. The cognitive test is configured to test the cognitive readiness of a person to engage in a particular activity. In some embodiments, the cognitive test measures the cognitive marker of a reaction time of a user between the presentation of a stimulus in a user interface on the user client 31 and the time when the user submits an input or response on the user client. At step 13 the measured reaction time is compared to a predefined acceptable reaction time or range of acceptable reaction times to determine whether the user is cognitively ready to engage in a given activity or whether the user is cognitively impaired. If the measured reaction time is equal to the acceptable reaction time or within the predefined range of acceptable reaction times, then the readiness determination function 10 will go to step 15 where the user will be allowed to proceed toward accessing the target 32. In some embodiments, at step 15, the user is allow to continue access previously granted.

In some applications at step 15, the user will be allowed direct access to the target computing resource 32. In some applications, there may be one or more other authentications or steps to be completed by the user before the target 32 provides the user will access to the target 32 after the determination system proceeds to step 15. In some embodiments, the determination system will prompt the user to submit login credentials, such as user name and password, before presenting the cognitive test at step 16. In some embodiments, the determination system will present the cognitive test at the same time as requesting the user to submit login credentials via the user client. In some embodiments, the determination system will present the cognitive test first, and if passed, the determination system will, at or after step 15, request the user to submit login credentials.

If at step 14, the reaction time is not equal to or within the pre-defined range of acceptable reaction times for accessing the target 32, the system will provide a failure notice, such as is provided in user interface 95 of FIG. 15, at step 16.

When the user fails to pass the cognitive test, the cognitive authority or other component of the determination system will deny the user access to the target computing resource. In some embodiments, the system is configured to allow the user to re-take the cognitive test a one or more predefined number of times after a failure. If the subsequent attempt(s) to pass the cognitive test is successful, the system may allow the user to access the target computing resource 32. In some embodiments, the system only allows the user to take the cognitive test once.

In some embodiments, at step 16, the system may also send notice of the failure to another person or location, such as a supervisor or a human resource department. The notice to another person or location may be sent by a message, such as email, SMS text, or other written communication or audio communication. The failure notice to the other person or location may include one or more of the following failure information: the user name of user that took the cognitive test, the reaction time, the date and/or time the cognitive test was taken, the location where the test was taken, and the user client identifier (e.g. a MAC address, IP address, computer name, electronic device name, and/or other identifying information or attribute).

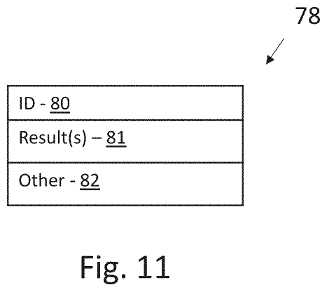

In some embodiments, the results of the cognitive test are stored in a data store 30a of or associated with or in communication with the cognitive authority 30. The cognitive test results may be stored in the data store 30a in any format capable of being read and/or updated. In some embodiments, the cognitive test results are stored in a user record 78, shown in FIG. 11, of the data store 30a. In some embodiments, the record 78 comprises an ID section 80, a result(s) section 81, and an other section 82. In some embodiments, the data store 30a is a database.

In some embodiments, the ID section contains an identifier that corresponds to the user. The identifier may be a unique identifier. The results section 81 may contain the past results, either in summary form, such as an average, or all or some individual past cognitive test results. The current results of the cognitive test at steps 12 and 13 can be saved to the results section 81. Further, the results section 81 can contain some or all of the failure information for each failure.

In some embodiments, a failure notice is not sent to another person, but instead, a list of failures can be retrieved by an administrator user of the cognitive authority by requesting, such as by querying the data store 30a, from the cognitive authority a list of users having one or more failed cognitive tests.

In some embodiments, instead of denying access at step 16 upon a user's failure to pass a cognitive test(s), the cognitive authority will log the test failure result in the data store 30a and/or send notice to another person or location, such as a supervisor or a human resource department, but will not deny access to the sought target 32. In such embodiments, the access control function 10 may instead be a cognitive readiness logging and/or notification function.

In some embodiments, the cognitive authority will allow other users, such as an administrator user or supervisor user, to indicate that the user whom failed a test should be denied access after such other user receives notice of a test failure. If such other user indicates that the user whom failed the test should be denied access, then the cognitive authority or other component of the determination system will deny the user access to the target computing resource 32. In the case where another component of the determination system denies the user access to the target computing resource 32, the cognitive authority may send notice or instructions to such other component that the user should be denied access to the sought target.

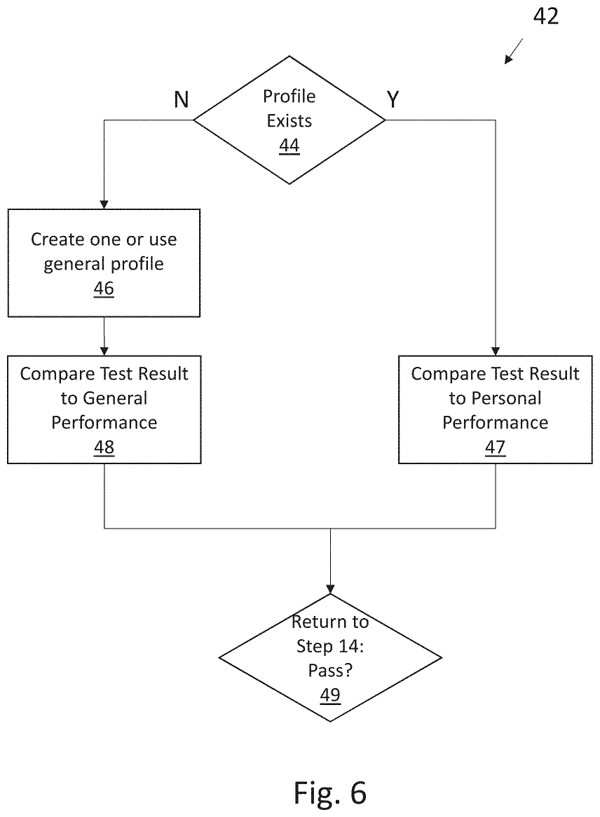

FIG. 6 shows one embodiment of a cognitive test result evaluation function 42 of step 13 of FIG. 1. At step 44, the cognitive authority checks the data store 30a to determine whether the data store has a profile for the user taking the cognitive test at step 12. The profile may be maintained in or comprise the user record 78 in the datastore 30a. In some embodiments, the cognitive authority compares a login credential, such as a user name, previously submitted to the ID section of the records in the datastore to determine whether the user has a profile/record in the datastore.

If a user profile doesn't exist, then at step 46 the cognitive authority will create a user profile in the data store 30a for the user. The creation of a user profile may involve creating a record 78 in the data store. In some embodiments, instead of creating a profile for the user at step 46, the cognitive authority will use a general profile which is not specific to anyone user.

At step 48, whether a user profile has been created or whether a general profile is used, the system will compare the cognitive test results of steps 12 and 13 to a predefined performance general standard of predefined range of acceptable reaction times. If the cognitive test results are within the predefined range of acceptable reaction times, then the cognitive authority will indicate that the user has passed to the cognitive test at step 49 and will return to step 14 of FIG. 1 to proceeding to step 15. At step 48, even if a user profile is created at step 46, that profile may not have specific prior test results corresponding to this user. Therefore a predefined general performance standard will be used at step 48. In some embodiments, the system may pre-populate the user profile with the general performance standard which will serve as the initial standards until the system receives a predefined sufficient amount of cognitive test result data from the user's tests results to customize that user's acceptable reaction time range.

If at step 44, a user profile exist at the cognitive authority for the user that is taking the cognitive test, then the cognitive authority will proceed to step 47, where the cognitive authority will compare the cognitive test results to the user's past personal performance reaction time standard. The past personal performance reaction time standard may be maintained in the results section 81 of the user record 78. If the results of the present cognitive test are within the personal performance reaction time standard associated with the user, such as provided in the results section 81 of the record 78 associated with the user, then the cognitive authority will indicate that the user has passed the cognitive test at steps 49 and 14. Then the cognitive authority will allow or indicate allowance of the user to proceed to step 15. The personal performance standard for a user can be a predefined range of acceptable reaction times.

When there is a user profile associated with the user, whether that profile exists prior to the user taking the test, or whether it's created at step 46, the cognitive authority 30 will save the results of the present cognitive test to the user profile, such as in the results section 81 of the user record 78.

In some embodiments, the cognitive authority will create and/or update the personal performance standard based on an average or mean of the test results of a prior predefined number of cognitive tests. For example, if the reaction times for the past four cognitive tests were 501 milliseconds (ms), 480 ms, 379 ms, 445 ms and the predefined number of tests was four then the average would be 451.25 ms. The cognitive authority may be configured to determine a predefined range based on the average by defining the range using a formula, such as plus or minus a predefined number or percent from the average. For example if the predefined range was plus or minus 10 percent of the average, and the average was 451.25 ms, then the predefined range would be 406.125 ms to 496.375 ms. If the predefined range was plus or minus 25 percent of the average, and the average was 451.25 ms, then the predefined range would be 338.43752 ms to 640.625 ms. The ranges may be inclusive or exclusive of the ends of the range.

In some embodiments, the cognitive authority will create and/or update the personal performance standard based on an average of the test results of the cognitive tests performed by the user during a predefined period of time. If the predefined time is within the last calendar month, and if the reaction times for the past four tests were 501 milliseconds (ms), 480 ms, 379 ms, 445 ms, but only the last three test were within the last month, then the average would be of 480 ms, 379 ms, and 445 ms for an average of about 434.67 ms. As explained above, the system may be configured to create a user predefined range based on the average by defining the range as plus or minus a predefined number or percent from the user's average.

In some embodiments, an administrator user of the cognitive authority may be allowed to define the extent of the predefined range. For example, an administrator user may be able to define that the predefined range is 10% of the average, or 25% of the average, or 20% of the average, or the like. In some embodiments, the cognitive authority may provide the administrator user a mechanism for adjusting the sensitivity of the test by expanding or retracting the predefined range of acceptable reaction times. And that expanding or retracting can be by adjusting the allowed percentage or value deviation from the average. The adjustment of the test sensitivity can adjust the sensitivity for personalized performance standards and/or general performance standards.

Specific groups of users may also be permitted a longer or shorter reaction time for a variety of reasons. For example, in some embodiments, the acceptable range may be modified by one or more demographic factors, such as age of the user or medical conditions that correlate with an increase in reaction time. Such modification may be automatically performed by the cognitive authority or may be set or adjusted by an administrator user of the cognitive authority. In some embodiments, the cognitive authority may be configured to provide older users with a predefined range of acceptable reaction times that comprises longer reaction times than the predefined range of acceptable reaction times for younger users. In some embodiments, the cognitive authority may apply a demographic shifting factor to extend or retract the personalized performance standards and/or general performance standards of acceptable reaction time ranges for a given user. In some embodiments, the demographic shifting factor may be added to or subtracted from the applicable acceptable reaction times for a user to provide the demographically adjusted range of acceptable reaction times. In some embodiments, the beginning and ending of the predefined range of acceptable reaction times are either or each multiplied by the demographic shifting factor and then the respective results are added to or subtracted from the beginning and ending of the predefined range of acceptable reaction times to provide the demographically adjusted range of acceptable reaction times.

In some embodiments, the cognitive authority determines the applicable personalized performance acceptable range of reaction times by determining the median or mode of a predefined set of prior cognitive test scores of the user, rather than using the average or mean. The applicable personalized performance acceptable range of reaction times may be a predefined deviation value or percentage from the determined median or mode, such as plus or minus 10 percent from the determined median or mode, or as otherwise explained above regarding acceptable deviations from an average.

In some embodiments, the cognitive authority will or is configurable to determine the acceptable range of reaction times based on a valid set of cognitive test results. As explained above, the valid set may be a predefined number of prior test results, such as four, or test results during a predefined time period, such as within the last month. However, the cognitive authority may consider other factors alone or in combination with a prior number of test, or a predefined timeframe, for determining which prior test results are included in a valid set. For example, the cognitive authority may be configured to exclude one or more outlier test results. If the prior five cognitive test results for a user were 500 milliseconds (ms), 480 ms, 5001 ms, 379 ms, 445 ms, the cognitive authority may be configured to exclude the 5001 ms result from the valid set due to its exceeding a predefined outlier threshold as compared to the other test results, when determining the acceptable range of reaction times for a user. In some embodiments, the outlier threshold will apply to one or more results that are more than 10 times higher than the longest other reaction times in a set of reaction times, however many other outlier threshold values may be used.

If at steps 47 or 48, the cognitive test results are not within the applicable range of acceptable reaction scores, then the cognitive authority will indicate that the user failed or did not pass the cognitive test at step 16, as explained above. At that time, as discussed above, one or more consequences of the failure will follow such as, denial of access to the target, logging of the cognitive test failure and related circumstances, and/or notification of another user, such as administrator user or supervisor user, whom may correspond to an appropriate authority within or outside an organization associated with the user taking the cognitive test.

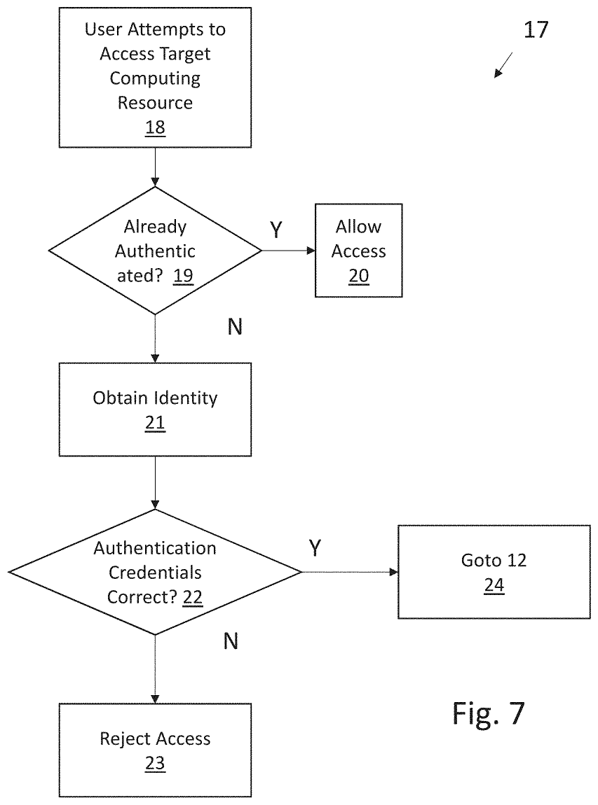

FIG. 7 shows an embodiment of the cognitive readiness determination system deployed in a login or access process or function 17. At step 18 the user attempts to access the target computing resource. In some cases, the attempt to access the target will involve the entry or submission of access credentials to the target. The target 32 may be configured to prompt the user to enter or submit access or authentication credentials. In some embodiments, access or authentication credentials are a username and password, biometric authentication credentials, two factor authentication codes, and/or the like.

At step 19 the target 32 determines whether or not the user is already authenticated. In some embodiments, this involves checking a cookie or other file stored on the user client to determine whether the user is already authenticated and allowed access at the present time. If the user is already authenticated and allowed access at step 19 then the system proceeds to allow the user to access the target at step 20 or to otherwise continue with previously granted access to the target at step 20. In some embodiments, the target 32 checks whether the user is already authenticated at step 19 before requesting access credentials.

If the user is not already authenticated at step 19, then the target 32 may optionally obtain identity information from an identity server 37 associated with the target 32 at step 21. The identity server may compare the user access credentials provided to one or more known credentials in an associated database. If the identity server finds a match with the user submitted credentials, then the user is a known user.

At step 22 the target determines whether the user has submitted and the determination system has received correct and valid authentication credentials. If so, then at step 24 proceeds to step 12 where the cognitive authority prompts the cognitive test and proceeds as discussed above from steps 12 to 15 or 16. If the determination system determines that the user has not submitted correct and valid authentication credentials at step 22 then at step 23 the target will reject or refuse access of the user.

In some embodiments, the login or access process or function 17 involves the single sign on authority 34. In such embodiments, the target 32 relies on the SSO authority 34 to determine whether the user has submitted the correct authentication credentials at step 22. In some embodiments, when the user is not already authenticated, the SSO authority will request from the user authentication credentials, such as a user name and password, at or before step 22, and then will determine whether the user has submitted the correct authentication credentials at step 22. It may determine whether or not the user has submitted the correct authentication credentials by comparing the received authentication credentials to authentication credentials stored by the SSO authority and associated with a user. The SSO authority will indicate to the target 32 whether or not the user has submitted valid and correct authentication credentials. Then the target will proceed to reject access at step 23 or proceed to cognitive testing at step 24 depending on whether the user provided valid and correct authentication credentials.

In some embodiments, user is prompted to take the cognitive test during or after a multi-factor authentication (MFA) process. The MFA process may be controlled or implemented by the target, the SSO authority 34, or a MFA authority 35. As the MFA process may be controlled or implemented by the target or the SSO authority, in some embodiments, the functions of the MFA authority 35 are functions of the target and/or the SSO authority.

In some embodiments, after the user submits a first set of authentication credential(s), such as a user name and password, the MFA authority will request the user to submit a second one or more authentication credentials or second action. The second authentication credentials may comprise a one-time password. The MFA authority will cause the one-time password to be sent to a second user client 31b known to be associated with the user. Therefore, the authentication process can involve authentication credentials that the user knows, e.g. the first set of authentication credentials, and authentication credentials that the user has, e.g. the one-time password sent to the second user client 31b known to be associated with the user. If the applicable correct one-time password is entered, the cognitive test will be presented to the user. In some embodiments, the one-time password is valid only for a limited time period.

In some embodiments, instead of sending a one-time password, the user client or second user client prompts the user to confirm the authentication by taking an action, which may be a second action, on the user client or second user client, such as selecting a user interface element, such as an “ok” button. The user client or second user client then sends confirmation to the MFA authority of the second action. When confirmation of the second action is received, then the cognitive test will be presented to the user.

The cognitive test may be presented to the user, and the user may take the cognitive test on the second user client 31b or the user client 31. In some embodiments, the second user client 31b and the user client 31 are the same device or are on the same device.

In some embodiments, the cognitive test is presented to the user, and the user takes the cognitive test, before the MFA authority sends the one-time user password or prompts the user for the second action.

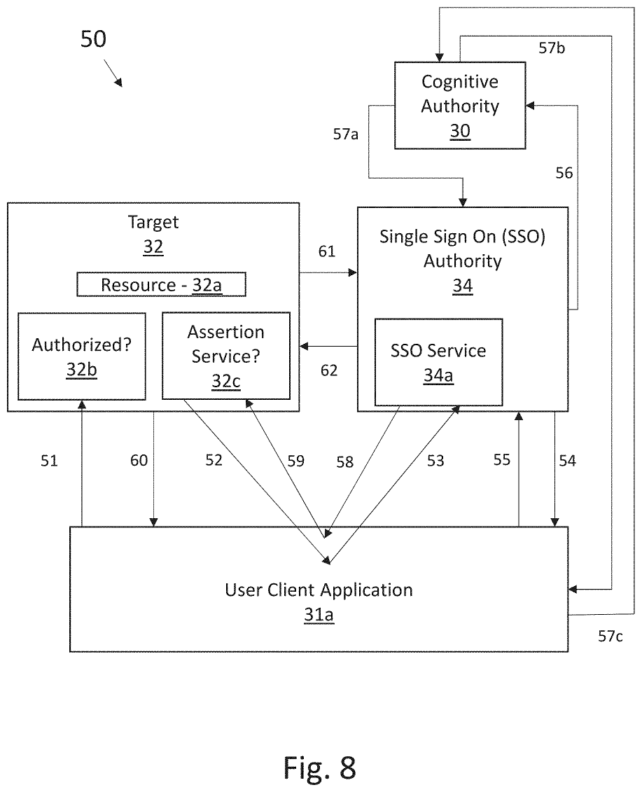

In some embodiments, the cognitive readiness determination system is implemented in or comprises a single sign-on environment 50. FIG. 8 shows an exemplary communications flow in a single sign-on environment 50 involving the user client 31. The environment 50 shows communication with a user client application 31a. In some embodiments, user client application is or includes a web browser or web browser functionality. In some embodiments, the client application is one or more other applications.

At step 51 the user client attempts to access the target 32, which may include access to a sub-resource 32a of the target computing resource 32. In some embodiments the attempt to access the target computing resource 32 involves attempting to access a predefined uniform resource locator (URL), associated with the target computing resource 32, with the web browser. In some embodiments, the attempt to access the target computing resource 32 involves opening or using an application on the user client.

The target computing resource 32 checks 32b if the user has a valid login session or security context for the target computing resource 32. In some embodiments, a valid login session or security context indicates whether the user or user client is already authenticated for the target and that such authentication has not expired. In some embodiments, the check involve accessing stored data, such as in a file, on the client device.

If the user does not have valid login session or security context, then the target computing resource 32 may save the location indicator, such as URL, of the resource that the user was attempting to access, for example in local state information. The local state information, or reference to it, can be saved, and optionally encoded, in a state relay value that is included with an authentication request.

Then at step 52, the target 32 sends an authentication request back to the client application 31a with a redirect instruction to the SSO authority 34. In some embodiments, the target 32 has an assertion service 32c that sends the authentication request back to the client application 31a with a redirect instruction. The redirect instruction may be or comprise a script code that will cause the client application 31a to automatically post/send the authentication request to the SSO authority.

In some embodiments, the target 32 sends the authentication request is in an HTML form. The HTML form may have the redirect script code and the authentication request. In some embodiments, the user will need to take action to cause the completion of the redirect to the SSO authority. Whether by user action or by automatic redirect, at step 53 the client application 31a will send the authentication request to the SSO authority. In some embodiments, the browser will issue a HTTP post request to send the HTML form comprising the authentication request to the SSO authority.

In some embodiments, the SSO authority 34 comprises an SSO service 34a that processes sign-on requests. When the SSO authority receives the authorization request from the client application 31a, the SSO authority determines whether the user has an existing login security context at the SSO authority, which meets the default or requested authentication policy requirements. If not, the SSO authority interacts with the client application 31a to challenge the user to provide valid authentication credentials. At step 54, the SSO authority sends a request to the client application for the user to submit valid authentication credentials. At step 55, the user causes the client application 31a to send authentication credentials for consideration by the SSO authority. If the SSO authority determines that the provided authentication credentials are valid, the SSO authority proceeds to step 56, where it then requests the cognitive authority present a cognitive test.

In some embodiments, the cognitive authority 30 will pass the cognitive test to the SSO authority 34 at step 57a and the SSO will pass the cognitive test to the client application 31a along step/path 54. The results of the cognitive test will be passed back to the SSO authority along step/path 54, and back to the cognitive authority along path 56. Therefore, the SSO authority will be an intermediary between the browser and the cognitive authority. In some embodiments, the SSO authority comprises the cognitive authority 30, as shown in FIG. 9A, and the SSO authority carries out the cognitive authority functions and exchanges with the client application 31a. In some embodiments, the cognitive authority presents the cognitive test directly to the browser at step 57b and receives the results from the browser at step 57c. In some embodiments, if a user is known to have successfully passed the cognitive test in a pre-defined authorized timeframe, the determination system will bypass the cognitive testing so as not to impact user experience. In some embodiments, the pre-defined authorized timeframe is 4 hours, 8 hours, 16 hours, 24 hours, 1 day, or another time period. In some embodiments, the cognitive authority has a function to allow an administrator to set the pre-defined authorized timeframe.

After receiving the results of the cognitive test, the cognitive authority evaluates the test results as discussed above, such as by the cognitive test result evaluation function 42.

If the SSO authority received valid authentication credential(s) from the user via the client application 31a and if the cognitive authority determined that the cognitive test results from the user via the browser were acceptable, then the SSO Authority creates a local login security context for the user, which effectively indicates a user is permitted access to the target 32 or target resource 32a.

In some embodiments, the cognitive test is sent by the cognitive authority or SSO authority to the client application 31a at step 54 with or at the same time as the SSO authority sends a request to the client application 31a for the user to submit valid authentication credentials. The client application 31a can then return the authentication credentials with or before returning the results of the cognitive test. The client application 31a may be instructed to request the user's authentication credentials before or after the cognitive test. The client application 31a may be instructed to present the request for authentication credentials on the same screen view as the cognitive test.

If the authentication credentials provided by the user are valid and a response time of the user to the cognitive readiness test was acceptable, then at step 58, the SSO authority will send a response message to the client application 31a indicating the user is authorized to access the target 32, and the client application 31a, at step 59, will redirect that message to the target 32. The response message comprises an assertion representing the user's logon security context.

In some embodiments, the assertion and/or the message is digitally signed. The digital signing can include encrypting a hash value of the assertion and/or message using a private key of the SSO authority that is associated with a public key of the SSO authority. The target 32 can use the public key of the SSO authority to decrypt the hash value and compare a hash of the assertion and/or message. If the decrypted hash value does not match a hash of the assertion and/or message, then the assertion and message will be rejected by the target 32 as invalid and will deny the user access to the target 32. A mismatch between the decrypted hash value does not match a hash of the assertion and/or message indicates that the assertion or message was changed after it was digitally signed and therefore cannot be trusted.

In some embodiments, response message is then placed within an HTML form, which may be as a hidden form control. If the SSO Authority received a relay state value from the target 32, the SSO authority will return the relay state value unmodified to the target 32, optionally in a hidden form control titled relay state in the HTML form. In some embodiments, the SSO authority sends the HTML form back to the client application 31a, at step 58, in the HTTP response. In some embodiments, the HTML form typically will be accompanied by script code that will automatically redirect or post the HTML form to the target at step 59. Alternatively, the user may have to take an action in the client application 31a to cause the redirect or completion of the redirect back to the target at step 59.

Then the target 32 determines whether the user or user client has the correct and valid authorization to access the requested target computing resource, such as resource 32a, based on the response message and the security context in the response message. If the user/user client has the correct and valid authorization to access the requested target computing resource, the resource 32a or access to it is then returned or provided to the client application 31a at step 60 by the target 32.

In some embodiments, instead of returning the response message from the SSO authority via the client application 31a, the response message is delivered directly to the target 32 from the SSO authority 34, such as shown at step 62. In this embodiment, the SSO authority sends an artifact to the client application 31a at step 58, which is redirected to the target 32 at step 59. When the target 32 receives the artifact, the target will, at step 61, send the artifact to the SSO authority. In response, the SSO authority will send the response message, at step 62 to the target 32.

In some embodiments, the SSO authority delivers the response message directly to the target 32 without an artifact request from the target and without sending an artifact to the client application.

In some embodiments, the user client 31 or the user client application 31a may not communicate directly with the target 32, the SSO, or the cognitive authority, but instead may communication through an intermediary. The intermediary may be a proxy server. The communications between the client application 31a and the target 32, the SSO authority 34, or the cognitive authority 30 may instead be between the intermediary and the target 32, the SSO authority 34, or the cognitive authority 30, as applicable. In some embodiments, the initial request, such as at step 51, that originates from the user client 31 or the user client application 31a, goes to the intermediary, and then to the target 32. All subsequent steps 52-55, 58 and 59 are between the intermediary and the applicable respective target 32, the SSO authority 34, or the cognitive authority 30. Then, if authorized, access to the resource is provided at step 60 to the user client.

In some embodiments, one or more of the cognitive authority 30, the target 32, and/or the SSO authority 34 comprise an identity store 30d, 32d, 34d, as shown in FIG. 9B. The identity stores 30d, 32d, 34d comprise user identity information, such as username among other identity data. In some embodiments, two or more of the cognitive authority 30, the target 32, and/or the SSO authority 34 have shared user identity information, so that at least some identity information between two or more of the cognitive authority 30, the target 32, and/or the SSO authority 34 are the same.

In some embodiments, one or more of the identity stores link the identity of a user in one system with the identity of a user in another. For example, 9C shows a portion of the identity stores 30d, 32d of the cognitive authority 30 and the target 32, respectively. The local ID dsmith in the identity store of the cognitive authority is associated, via the linked ID 30243, with the local ID of Dan in the identity store of the target 32.

In some embodiments, one or more of the identity store of the target 32, SSO authority 34, and/or the cognitive authority 30 are not linked. For example, the target 32 or the SSO authority 34 could use transient identifiers that only correspond to the identity of a user in the target 32 for one session or one time period. The transient identifiers may change on a regular interval or may be one-time use identifiers. In this way, in some embodiments, the one-time use identifier will not allow the cognitive authority to compare the results of the current test results to prior test results of a user because of the changing one-time-use identifiers. Further, the cognitive authority may not know the actual identity of the user taking the cognitive test other than by the transient identifier. This configuration may allow more private or anonymous use of the cognitive authority's cognitive test and access control. The increased privacy allowed by transient identifiers or persistent identifiers that are not linked to the user's profile or known to the cognitive authority, may be desired or sought by certain users or use cases. In the case of transient identifiers, the cognitive authority will compare the current test results to a general accepted result or range of results to determine if the cognitive test results are acceptable.

In some embodiments, the single sign-on environment uses the OASIS Security Assertion Markup Language (SAML) standard, providing an XML-based framework for describing and exchanging security information between on-line entities, including between the target 32, the user client 31, and/or the SSO authority. Therefore, in some embodiments, the communications/messages at steps 51, 52, 53, 54, 55, 58, 60, 61, and 62 are in SAML format or conform to the SAML or SAML version 2.0 standard.

In some embodiments, the SSO authority 34 is configured to control access and provide authentication services to multiple computing resources. In some embodiments, the SSO authority is configured to control access not only to the target 32 and the resource 32a of the target, but also to other computer systems and computing resources. In some embodiments, the SSO authority 34 is capable of controlling access and providing authentication services to 2, 3, 4, 5, 6, 7, 8, 9, 10, or more computer systems or computing resources.

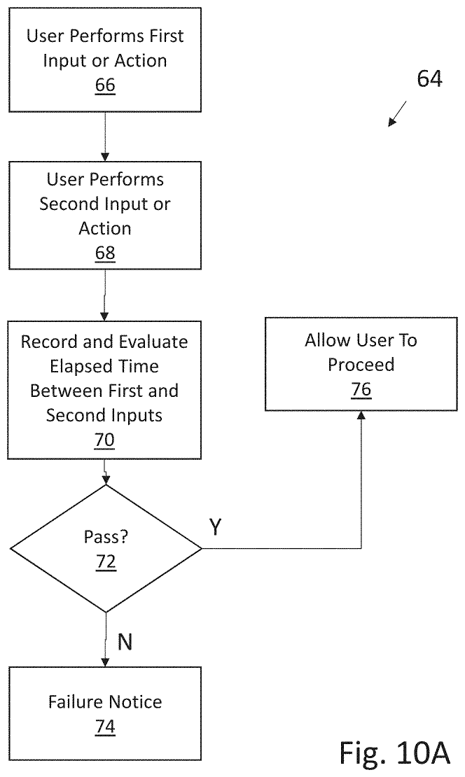

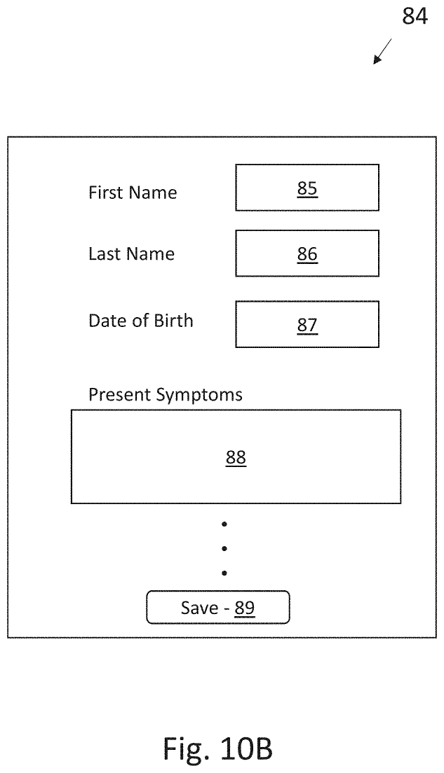

FIG. 10A shows a flow diagram of a second embodiment access control function 64 of the cognitive readiness determination system. At step 66 the user performs a first input or action at the user client. At step 68 the user performs a second input or action at the user client. At step 70 the user client 31 determines the elapsed time between the first and second input or action. The cognitive authority receives the elapsed time from the user client and determines whether the elapsed time matches an acceptable elapsed time or is within a range of acceptable elapsed times. The cognitive authority 30 will signal to the target that the elapsed time is or is not acceptable. Then if the elapsed time is acceptable the target will allow the user to proceed or continue at step 76, with or without giving the user notice of a successful elapsed time, access to the target that was previously granted. If the elapsed time is not acceptable, the cognitive authority or the target 32 will provide the user with a failure notice at step 74, such as by displaying a failure message on a user interface of the user client. At step 74, the target 32 may discontinue the user's access to the target computing resource 32 and/or may send notice to another user, such as supervisor, and/or may record/log the failure and data corresponding to the failure. In some embodiments, another component of the determination system denies the user access or discontinues access to the target computing resource 32 and the cognitive authority may send notice or instructions to such other component that the user should be denied or discontinued access to the sought target.

The access control function 64 may instead be a cognitive readiness logging and/or notification function where at step 72 upon a user's failure to pass a cognitive test(s), the cognitive authority logs the test failure result in the data store 30a and/or sends notice to another person or location, such as a supervisor or a human resource department, but does not deny or terminate access to the target computing resource 32.

In some embodiments, the first and second input or action, is an input or action that the user regularly undertakes in connection with the target computing resource 32. In some embodiments, such input or action includes any of entering, changing, and/or deleting information in a given one or more fields or areas of a user interface, and/or selecting clicking on, moving, changing, or otherwise interacting with one or more user interface element(s) of a user interface or elements of the user client, and/or requesting access to data, including to view an image or other piece of information or data. An acceptable elapsed time or range of acceptable elapsed times may be saved in the results section 81 of the user record 78.

FIG. 10B shows an exemplary user input interface 84. The interface 84 comprises a plurality of input fields 85, 86, 87, 88 and at least one selectable button 89. The input fields comprises a first name field 85, a last name field 86, a date of birth field 87, and a present symptoms field 88. The vertically extending ellipsis dots indicate that the interface can have additional fields, either below the symptoms field or elsewhere on the interface 84. The second embodiment readiness determination function 64 can be configured to measure the elapsed time between the time the user client receives input from a user in any one of the input fields 85, 86, 87, 88 or selecting the button 89 and the time when the user client receives input in any other of the input fields 85, 86, 87, 88 or selecting the button 89. For example, the first input or action at step 66 can be a user inputting data into any of the input field 85 and the second input or action at step 68 can be the user inputting data in field, such as field 86. The elapse time between the first input and the second input can be measured and evaluated as explained regarding function 64. In some embodiments, the user input interface 84 may have more or fewer input fields, buttons or other data input areas or action areas, which may be arranged in a different manner than is shown in interface 84.

Similar to the past personal performance reaction time standard described regarding FIG. 6, in some embodiments, the cognitive authority will create and/or update the personal performance elapsed time standard based on an average of the user's elapsed time between one or more prior instances of the user providing the given first input or action and given second input or action. For example, if the elapsed time for the past four instances of the user providing the given first input or action and a given second input or action was 1000 milliseconds (ms), 1100 ms, 1200 ms, 1050 ms and the predefined number of tests was four then the average would be 1087.5 ms. The cognitive authority may be configured to determine a predefined range based on the average (or average of a valid set of prior elapsed times) by defining the range as a formula, such as plus or minus a predefined number or percent from the average of a valid set. For example if the predefined range was plus or minus 10% of the average, and the average was 1087.5 ms, then the predefined range would be 978.75 ms to 1196.25 ms. If the predefined range was plus or minus 25% of the average, and the average was 1087.5 ms, then the predefined range would be 815.625 ms to 1359.375 ms. The ranges may be inclusive or exclusive of the ends of the range. As explained above regarding past personal performance reaction time standard described regarding FIG. 6, instead of basing the personal performance elapsed time standard based on an average of a set of prior elapsed times, the personal performance elapsed time standard could be based on a median or mode of a set of prior elapsed times. Further, as explained above regarding past personal performance reaction time standard described regarding FIG. 6, the personal performance elapsed time standard could be based on a valid set of prior elapsed times.

In some embodiments, the system will create and/or update the past personal performance reaction time standard based on an average of the test results of the cognitive tests performed by the user during a predefined period of time. As explained above, the system may be configured to create a past personal performance reaction time standard range based on a user average by defining the range as plus or minus a predefined number or percent from the user's average.

FIG. 12 shows a first embodiment cognitive readiness test user interface 90 of the cognitive readiness determination system where a cognitive test 91 can be presented to a user and where the user can interact with the cognitive test. The user interface is a graphical user interface. The user interface comprises a dial 92. The dial comprises a track 100. The track comprises a starting end 96 and a terminal end 98. A goal element, such as a goal bar 102 intersects the track 100. Extending radially from the track are a plurality of hash marks 105. Interior of the track is a status display 108. Below the status display 108 is a reaction time display 110. Below the reaction time display 110, is a scoreboard 94.

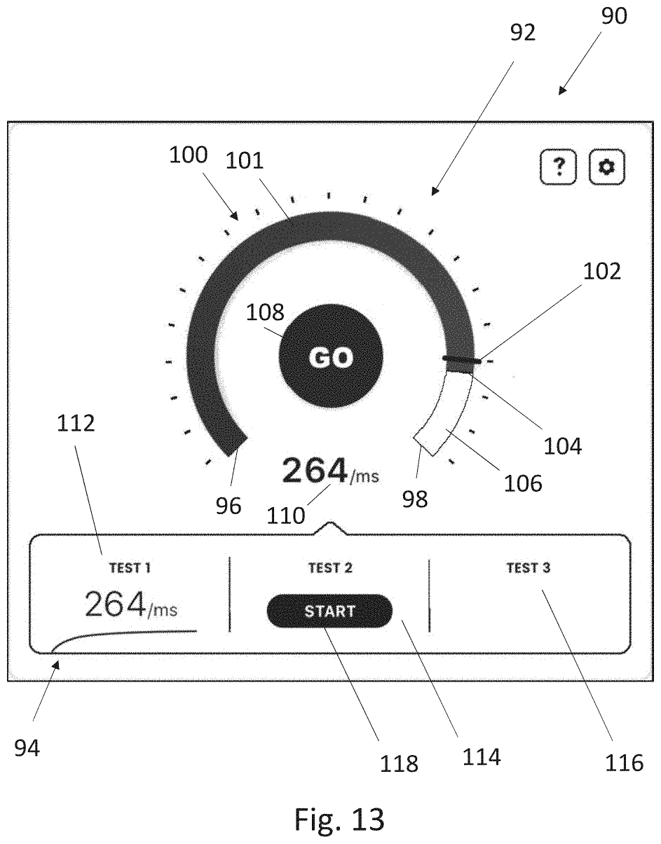

In some embodiments, the scoreboard has three areas 112, 114, 116 corresponding to three tests respectively. The scoreboard provides a start button 118 below the corresponding test. The user can begin the test by selecting the start button that is presented below the test in the scoreboard. When the corresponding test is complete the reaction time results for that test will be displayed in the location of the start button 118 as is shown in FIG. 13, and the start button will then appear below the next test to be performed. In the case of FIG. 13 the next test to be performed is test number two. When all tests have been completed the start button will no longer appear in the scoreboard, instead the corresponding scores will be shown below the respective tests.

In some embodiments, the user starts the cognitive test by selecting the start button on the scoreboard. After selecting the start button on the scoreboard, the user client 31 will provide the user with a countdown to the start of the test. The countdown may be in the form of the presentation of the following numbers sequentially one after the other: 3, 2, 1, in the status display 108. After the last countdown number is displayed, an indication that the test is started will be displayed. That indication may be in the form of the word “GO” as shown in FIG. 13. When the test begins, the track will fill with a progress bar 101 as shown in FIG. 13. The progress bar 101 will begin filling the track from the beginning 96 and will move toward the end 98. And the leading face 104 of the progress bar will move from the beginning 96 toward the end 98.

In some embodiments, the user is instructed to stop the progress of the progress bar 101 so that the leading face 104 is as close as possible to the goal bar 102 by providing one or more predefined input(s). Such inputs may include hitting a key, touching a touch screen, or clicking a mouse button. Therefore, the user should attempt to stop the progress of the progress bar so that the leading face 104 is on or co-planar with the goal bar 102. In the example shown in FIG. 13, the leading face has been stopped slightly passed the goal bar 102.

An end portion 106 of the track 100 is shown in FIG. 13 to be the portion of the track on which the progress bar has not traveled. The user client 31 then measures the time between when the leading face 104 reached the goal bar 102 and when user client 31 received the predefined input from the user. In the example shown in FIG. 13, the delay between when the leading face 104 reached the goal bar 102 and when user client received the predefined input from the user was 264 milliseconds (ms). Therefore the user's reaction time for test number 1 was 264 ms.

In some embodiments, as shown in FIGS. 12 and 13 the user client is configured to present more than one cognitive test during a testing session. FIGS. 12 and 13 show that the cognitive authority will cause the user client to present three cognitive tests. In other embodiments, any number of cognitive test may be presented in the user interface 90. When the user selects the start button 118 for test number two, the progress bar 101 will reset and the leading face 104 will begin from the start end 96 when the test number 2 starts. The same reset to the start end 96 will occur at the beginning of test number 3.

In some embodiments, the cognitive authority 30, will take an average of the number of tests taken at a testing session. In the example of FIGS. 12 and 13, the system will take an average of the three test scores and compare that average to the personal performance standard of the user or the general performance standard as described regarding FIG. 6 to determine whether the user has passed the cognitive test.

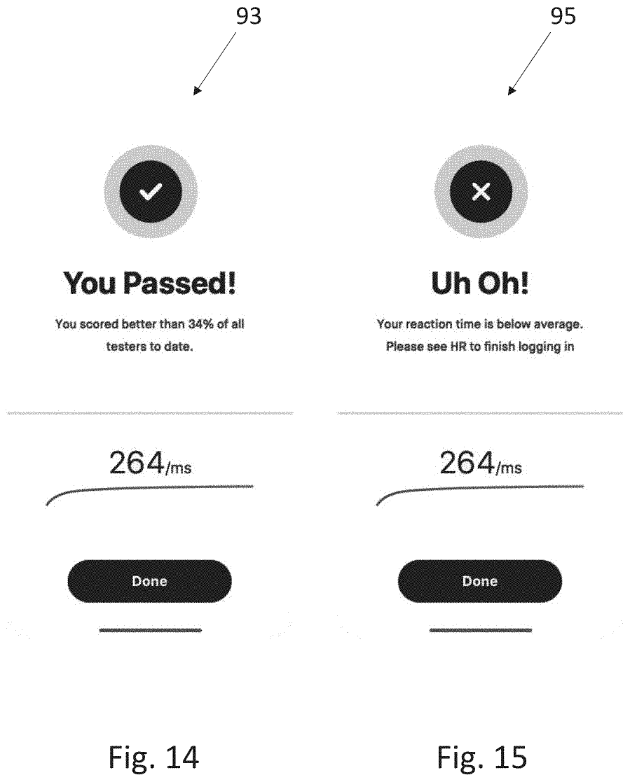

FIG. 14 provides an exemplary cognitive test pass user interface 93, which shows that the user has passed. In some embodiments, the user interface 93 may comprise a checkmark and a text indication that “you passed.” In some embodiments, the user interface 93 may comprise an indication of the user score relative to other users who have taken the cognitive test, as a percentage. For example the user interface 93 comprises the text “you scored better than 34% of all testers today.” In some embodiments, the user interface may comprise an indication of the user score relative to the user's prior scores from taking the cognitive test on prior occasions. The user interface 93 may comprise one or more of the test scores resulting from the tests that the user just took or an average of those tests. For example, 264/ms is shown in FIG. 14.

FIG. 15 provides an exemplary cognitive test fail user interface 95, which shows that the user has not passed. In some embodiments the user interface 95 comprises an X and a text indication that the user has not passed the cognitive test. The user interface 95 may comprise instructions such as the instruction to visit human resources or the user's supervisor or another person or department or location. The user interface 95 may comprise one or more of the test scores resulting from the tests that the user took or an average of those tests. For example, 264/ms is shown in FIG. 15.

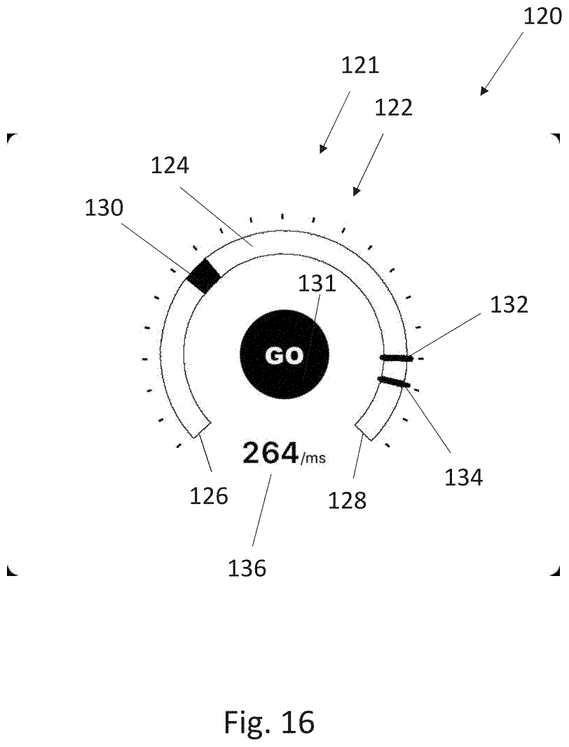

FIG. 16 shows a second embodiment cognitive readiness test user interface 120 where a cognitive test 121 can be presented to a user and where the user can interact with the cognitive test. The user interface comprises a dial 122. The dial comprises a track 124. The track comprises a starting end 126 and a terminal end 128. A goal area start indicator, such as a start bar 132 and a goal area end indicator, such as end bar 134 each intersect with the track 124. Extending radially from the track are a plurality of hash marks. Interior of the track is a status display 131. Below the status display 131 is a reaction time display 136. Optionally, below the reaction time display 110, is a scoreboard (not shown).

When a user selects to start the cognitive test of FIG. 16, a user interface element such as a track block 130 will begin from the beginning end 126 of the track and will travel along the track towards the terminal end 128. The track block 130 does not fill the entire track as it passes over the track, but instead the track bar has a discrete length. The track block fills a portion of the track, as shown in FIG. 16, as the track block moves from the beginning 126 toward the terminal end 128.

The user may be instructed to stop the progress of the track block 130 so that the track block is between the start bar 132 and the end bar 134 by providing one or more predefined input(s). The user client then measures the time between when track block 130 is within the start and end bars 132, 134 and when user client received the predefined input from the user. In the example shown in FIG. 16, the delay between when the time between when track block 130 is within the start and end bars 132, 134 and when the user client received the predefined input from the user via the user client was 264 milliseconds (ms). Therefore, the user's reaction time for the cognitive test was 264 ms.

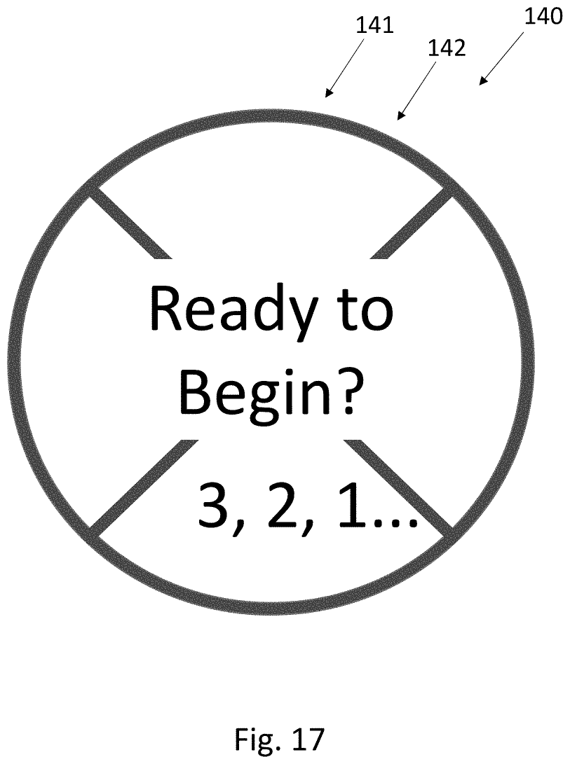

FIGS. 17 through 29 shows a third embodiment cognitive readiness test user interface 140, 143, 158 where a cognitive test 142, in various stages of progress, is presented to a user and where the user can interact with the cognitive test.

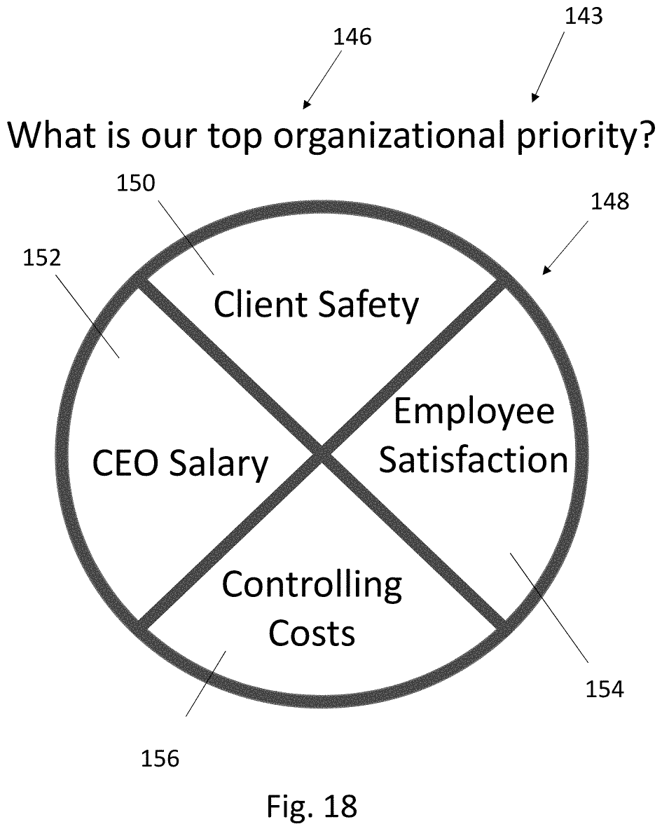

In FIG. 17 the user interface comprises a wheel 141. When the user indicates she/he would like to proceed with the test by providing one or more predefined inputs to the user client, the user client will provide the user with a countdown to the start of the test. The countdown may be in the form of the presentation of the following numbers sequentially one after the other: 3, 2, 1, in the status display 108. After the last countdown number is displayed, the interface 143 of FIG. 18 is shown.

The user client, as directed by the cognitive authority, is configured to display a question 146, such as “what is our top organizational priority?” and also to display two or more possible answers. In some embodiments, the possible answers are presented in a divided wheel or pie configuration 148. The wheel 148 may comprise a plurality of possible answer areas 150, 152, 154, 156 corresponding to possible answers. FIG. 18 shows the possible answers as “client safety” in answer area 150, “CEO salary” in answer area 152, “controlling costs” in answer area 156, and “employee satisfaction” in answer areas 154.

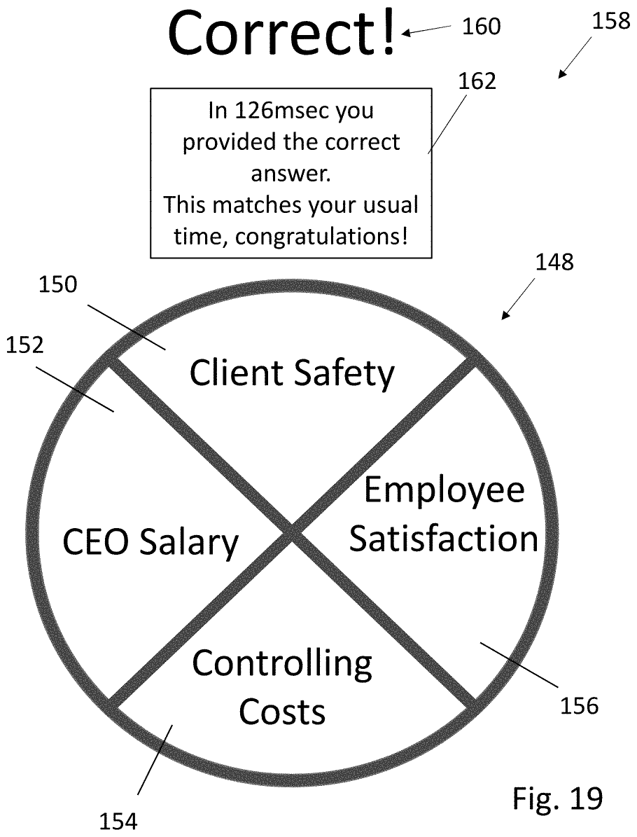

The user is to select at least one of the possible answers that best answers the question 146. The user client is configured to determine if the selected answer is correct and to measure the answer reaction time between the time the question 146 is displayed to the user and the time the user selected an answer or answer area. In FIG. 19, the user selected answer area 150 corresponding to answer “Client Safety,” which was the correct answer to the question 146. The user client measured the answer reaction time at 126 ms and determined and cause the result display 160 of “Correct!” The cognitive authority compares that answer reaction time to either a general range of acceptable answer reaction times or to a user's personal performance reaction time/range for the user taking the test. The user's personal performance reaction time/range can be based on prior reaction times in answering questions 146 in a cognitive test, as explained above regarding the cognitive test result evaluation function 42. Reactions times can be saved in the results section 81 of the record 78 by the cognitive authority.

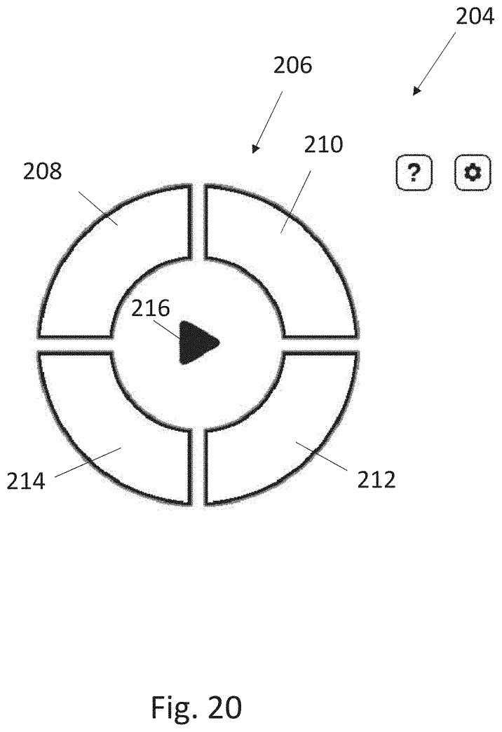

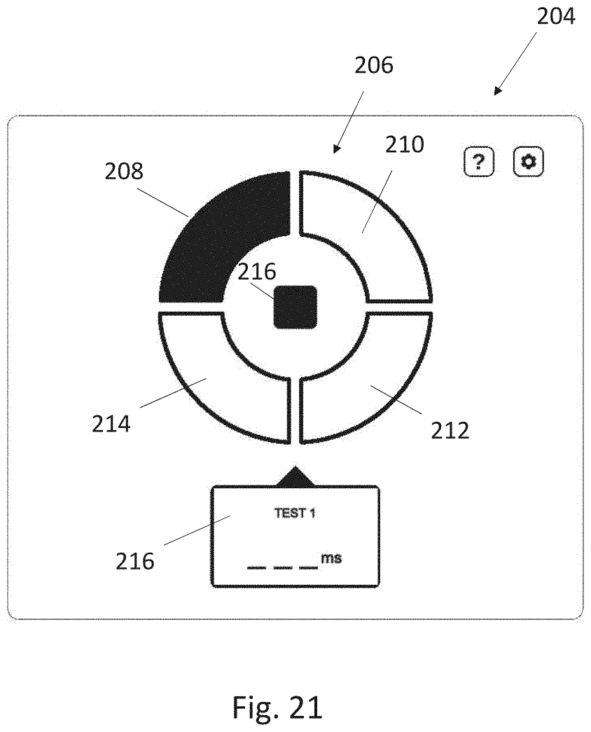

FIGS. 20 and 21 shows a fourth embodiment cognitive readiness test user interface 204 of the cognitive readiness determination system where a cognitive test 206 can be presented to a user and where the user can interact with the cognitive test. The interface 204 comprises a plurality of tiles 208, 210, 212, 214. The tiles are comprise a portion of a circle or a ring shape shown in FIG. 20. Interior of the tiles is a status indicator 216.

FIG. 21 show the interface 204 in a second stage of use where one of the tiles 208 is indicated. Indication may be considered a stimulus. The indication may include illumination, contrast change, or color change as compared to the level of illumination, contrast or the color of the tile in the first stage of use before the indication occurs, such as of FIG. 20. In some embodiments, the user client is configured to measure the reaction time between when one or more of the tiles is indicated and the time when a predefined user input is received. In some embodiments, after the predefined user input is received, the user client will display the reaction time in the score board 216 below the tiles.

As explained regarding user interface 90, the user client may be configured to prompt the user to take multiple test within a single testing session. For example, after the user submitted the predefined user input in response the indication of the tile 208, the user client may indicate the tile 210. The user client will the measure the time between when the tile 210 is indicated and when a predefined user input is received. Likewise, the user client may then indicate tile 212 and measure the reaction time between the indication of tile 212 and when a predefined user input is received. Similarly, the user client may then indicate tile 214 and measure the reaction time between indication of tile 214 and when a predefined user input is received. The user client may send the measured reaction time(s) to the cognitive authority for determining whether one or more of the measured reaction times is acceptable. The user client may be configured to indicate one or more of the tiles 208, 210, 212, 214 in any order in a series of test. In some embodiments, an individual tile may be indicated more than once during a testing session.

In some embodiments, in addition to or instead of measuring reaction time, the user client may be configure to measure whether the user has copied a sequence of indications. The user client may be configured to indicate the tiles in a predefined order. Then the user will select the tiles in the predefined order they were indicated. For example, if the tiles were indicated in the following order 210, 208, 214, 212. The user client would be configured to detect whether the user selected the tiles in the same order 210, 208, 214, 212. In some embodiment, the user moves a pointer or cursor to, and selects, each of the tiles in the order indicated to select the tile in the order indicated. In some embodiments, the user uses a touch screen of the user client to touch each of the tiles in the order indicated to select the tile in the order indicated.

In some embodiments, the user client is configured to measure the first reaction time between when the last indicating of the tiles and the time when a first tile is selected by the user. The user client will send the first reaction time to the cognitive authority to determine whether the first reaction time was acceptable.

In some embodiments, the cognitive authority is configured to control access to the target computing resource 32 based on the first reaction time and/or whether the order the tiles were selected matched to order that the tiles were indicated. Therefore the cognitive test may not be considered passed, at step 14, unless the order the tiles were selected by the user matched the order that the tiles were indicated. In some embodiments, the cognitive test may not be considered passed, at step 14, unless the order the tiles were selected by the user matched the order that the tiles were indicated and the first reaction time was an acceptable reaction time for the user, such as being within a predefined range of acceptable reaction times for the user. The cognitive authority may store the results of the tile selection to the results section 81 of the record 78 for the user.

The instructions and configurations for user client functionality of presenting, starting, stopping, and measuring the reaction time and/or other measures of cognitive function, such as presenting a sequence of indication and recording a sequences of inputs, of a user can be provided to the user client by the cognitive authority at or before the time when the cognitive test is to be presented on the user client. In some embodiments, the user client functionality of measuring the reaction time of the user can be performed by the cognitive authority with user inputs being passed from the user client to the cognitive authority and responses returning to the user client.

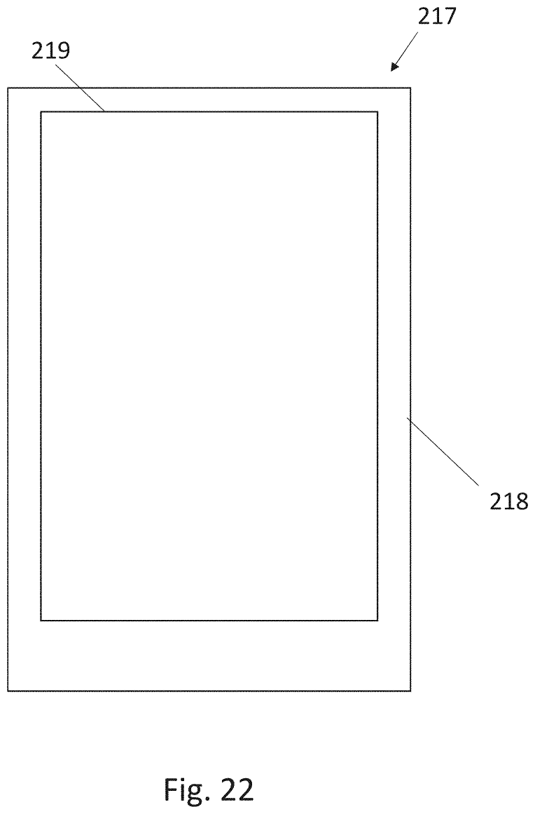

FIG. 22 shows a first embodiment housing 217 of an embodiment of the user client 31, 31b. The housing comprises a body 218 and a display 219, which may be a touch sensitive display. The housing may be usable with a mobile embodiment of the user client 31, 31b. The housing 217 may comprise other components not shown. Other housings are possible. In some embodiments, the housing 217 is a display housing of an embodiment of the user client.

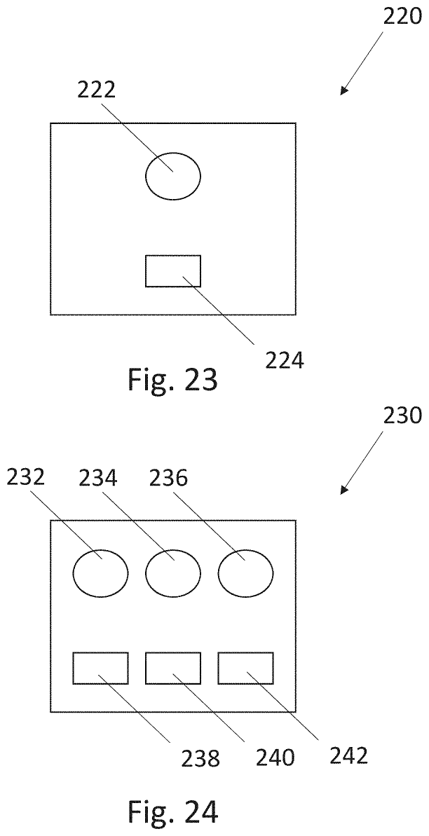

FIG. 23 shows a second embodiment housing 220 of an embodiment of the user client 31, 31b usable with the cognitive readiness determination system. In some embodiments, instead of or in addition to the presentation of the cogitative test on a graphical user interface, the cognitive test may comprise the use of an illuminating element and a push button. The housing 222 comprises at least one illuminating element 222, such as a light, which may be a light emitting diode (LED). The housing comprises a user input device 224, such as a push button. The user client is configured to receive instructions from the cognitive authority to begin a cognitive test or prompt a user to begin the cognitive test. The user client may be configured to illuminate the illuminating element 222 to start the test. The user client comprises a measuring mechanism to measure the reaction time between when the illuminating element is illuminated and the time when the button 224 is pressed. The measuring mechanism may be a timer that is configured to measure the elapsed time between the illumination the illuminating element 222 and the time when the button 224 is pushed. The user client will send the reaction time to the cognitive authority to determine whether the reaction time was acceptable. The user client may be configured to prompt the user to take multiple cognitive tests within a testing session.

The user client may be configured to indicate, by use of the illuminating element 222, to a user that the user should take the cognitive test. In some embodiments, this indication may be in the form of maintaining the illuminating element in an illuminated condition. In some embodiments, this indication may be in the form of flashing the illuminated element on and off. The user can indicate that the user is ready to take the cognitive test by pressing the button 224. After the user presses the button, the illuminating element will go out. Then, the user client will again illuminate the illuminating element and measure the time between when the illuminating element is illuminated when the button is again pushed by the user.

FIG. 24 shows a third embodiment housing 230 of an embodiment of the user client 31, 31b usable with the cognitive readiness determination system. The housing 230 comprises a first, second, and third illuminating elements 232, 234, 236 and a corresponding first, second, and third push buttons 238, 240, 242. The user client may be configured to illuminate the illuminating elements 232, 234, 236 in a predefined order and to determine whether the user has copied the order by pressing the corresponding push buttons in that predefined order. For example, if the illumination order is elements 234, 236, and 232 and the buttons are pressed in the following button press order 240, 242, 238, the user client recognizes that the user pressed the buttons in the correct order corresponding to the order that the illuminating elements were illuminated. The user client may recognize that the user pressed the buttons in the correct order by recording the order that the buttons were pressed and comparing the recorded order to the order that the illuminating elements were illuminated. In some embodiment, the user client transfers the order of button press inputs of the user to the cognitive authority and the cognitive authority determines whether the user has pressed the buttons in the correct order.

In some embodiments, the user client is configured to measure the first reaction time between when the last illuminating element was lit and the time when a button 238, 240, 242 is pressed by the user. The user client will send the first reaction time to the cognitive authority to determine whether the first reaction time was acceptable.

In some embodiments, the cognitive authority is configured to control access based on the first reaction time and/or whether the order the buttons 238, 240, 242 where pressed matched to order that the illuminating elements were illuminated. Therefore the cognitive test may not be considered passed, at step 14, unless the order the buttons where pressed matched the order that the illuminating elements were illuminated, and the first reaction time was an acceptable reaction time for the user, such as being within a predefined range of acceptable reaction times for the user. The cognitive authority may store the results of the button order press to the results section 81 of the record 78 for the user.

In some embodiments, access to the target computing resource 32 may allow the user access to any number of other resources, which may be physical and/or digital. For example, the target computing resource 32 may provide or control access to a restricted access physical space, area, or container. The target computing resource 25 may be or comprise or be connected to and control a linear actuator, such as a solenoid, that drives one or more a lock elements, such as lock bolt, between a locked position and a released position to releasably secure an access door or gate to a restricted access physical space, area, or container in a closed position. The lock element(s) can engage a recess of a frame adjacent the door or gate. In some embodiments, the area is a cabinet, room, building, or portion of a building. In some embodiments the restricted access area contains drugs, medicine, equipment, and/or hazardous items/materials. In some embodiments, the target computing resource 32 provides or controls access to, interaction with, or operation of a device or a machine, which may be a stationary machine or movable machine, such as a vehicle. In some embodiments, access to the target computing resource 32 can control or allow a user to begin or continue the user's efforts, such as work efforts, in whole or in part.

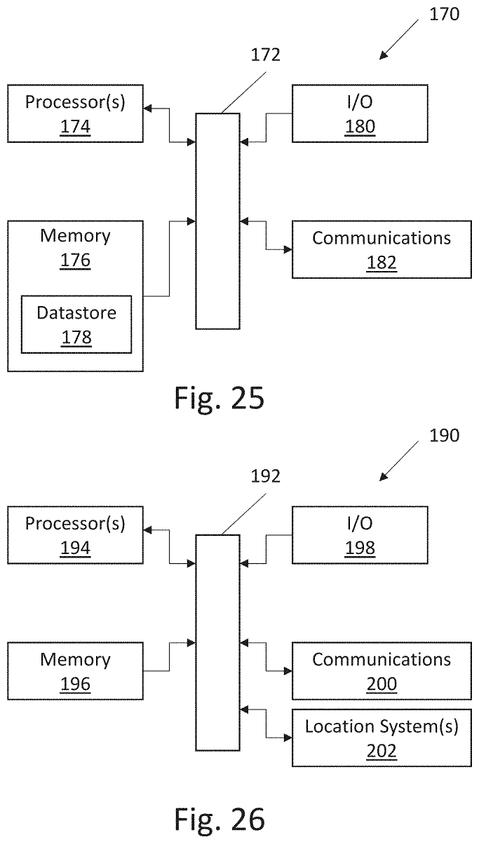

FIG. 25 shows a block diagram of an example server computer architecture 170 for implementing the features and processes described herein, such as in reference to the cognitive authority 30, SSO authority 34, target computing resource 32, critical system(s) 39 or to other server side functionality. Other architectures are possible, including architectures with more or fewer components. In some implementations, the architecture 170 comprises one or more communication channels 172, such as a bus that connects one or more processor(s) 174, a memory 176, one or more input device(s) 180, one or more output device(s) 180, and one or more communications interface(s) or system(s) 182. The one or more communication channels 172 allow the transfer of data, communications, and control signals between the various components connected to the channels 172.

The communications interface(s) or system(s) 182 may comprise wired or wireless network interfaces, such as an Ethernet wired network interface. The communication system may be configured to connect or synchronize with a client/host device using one or more protocols, such as HTTP, TCP/IP or other protocols. The input device(s) 180 may comprise a keyboard, a mouse, and/or a touch-sensitive display. The output device(s) 180 may comprise a display, such as an LCD display.

The memory includes non-transitory computer readable medium(s) that may comprise one or more non-volatile media, such as optical, magnetic disks, and/or flash memory, and/or volatile media, such as random access memory (RAM). The term non-transitory used herein is a limitation of the medium itself, in that the medium is tangible and not a propagating signal. But non-transitory is not a limitation on the data storage persistency. Therefore, non-transitory encompasses mediums that do not necessarily store information permanently, such as random access memory.

The memory may comprise a datastore 178. The datastore 178 may be or comprise the data store 30a or the identity store 30d, 32d, or 34d, in the case of cognitive authority 30, target resource/system 32, and SSO authority 34, respectively.

The computer readable mediums may comprise an operating system instructions executable by the one or more processor(s) 174 to perform operating system functions and operations, network communication instructions executable by the one or more processor(s) 174 to perform communications functions and operations, and the instructions executable by the one or more processor(s) 174 to perform the operations and functions of the cognitive authority 30, SSO authority 34, target 32, or critical system(s) 39, as the case may be. The operating system can perform tasks, such as managing files and directories on the computer readable mediums, managing traffic on the one or more communication channels 172, recognizing input from input devices 180, providing output to output devices 180, among other tasks. The network communications instructions can enable the establishing and maintain of network communications.

FIG. 26 shows a block diagram of an example user client architecture 190 for implementing the features and processes described herein, such as in reference to the user client 31, the second user client 31b, or to other client side functionality. In some implementations, the architecture 190 comprises one or more communication channels 192, such as a bus that connects one or more processor(s) 194, a memory 196, one or more input device(s) 198, one or more output device(s) 198, and one or more communications components or systems (s) 200. The one or more communication channels 192 allow the transfer of data, communications, and control signals between the various components connected to the channels 192.

The communications systems (s) 200 may comprise wired and/or wireless network interfaces, such as an Ethernet wired network interface. In some embodiments, the wireless interfaces comprise one or more of radio frequency transmitters and receivers and/or optical receivers and transmitters. In some embodiments, the communications system may be designed depending on the communication networks where the device is intended to operate. In some embodiments, the communication system is configured to operate using protocols, such as IEEE 802.x (e.g. WiFi), 3G, 4G, long-term evolution (LTE), 5G, near field communications (NFC), code division multiple access (CDMA), and/or global systems for mobile communications (GSM). The communication system may be configured to connect or synchronize with a host device using one or more protocols, such as HTTP, TCP/IP or other protocols.

The I/O system 180 may comprise input devices, such as a keyboard, a mouse, and/or a touch-sensitive display. The I/O system 198 may comprise output device(s) 198 such as a display 219, which may be an LCD display or a touch-sensitive display. When the display is a touch-sensitive display it is an input and an output device. The display is capable of displaying a graphical user interface, such as the user interfaces 90, 93, 95, 120, 140, 143m, 158, and 204.

In some embodiments, the I/O system of the user client computer architecture 190 comprises a touch controller in communication with a touch surface, which can be a touch sensitive display surface. In some embodiments, the touch surface and touch controller are configured to detect contact and movement or break of movement using one or more touch sensitive technologies. Touch sensitive technologies can include capacitive, infrared, resistive, and surface acoustic wave technologies, and or other components for determining one or more points of contact or break of contact with the touch surface. In some embodiments, the touch surface is configured to display a virtual keyboard.

In some embodiments, the architecture 190 comprises a location system(s) 202. The location systems may comprise a location processor. The location system may comprise functions for determining the location of the architecture. In some embodiments, the location system 202 comprises GPS functionality. The GPS functionality uses data/signals transmitted from satellites to determine location. In some embodiments, the location system uses other inputs, such as other signals, including Wifi signals, to determine a location or an estimated location of the architecture or to enhance the accuracy of the location determination. In some embodiments, the architecture comprises a memory interface between the memory 196 and one or more other components of the architecture.

The memory 196 includes one or more non-transitory computer readable medium(s) that may comprise one or more non-volatile media, such as optical, magnetic disks, and/or flash memory, and/or volatile media, such as random access memory (RAM).

In some embodiments, the computer readable mediums may comprise instructions executable by the one or more processor(s) for providing corresponding functionality, such as one or more of operating system instructions for facilitating operating system functionality, network communication instructions for facilitating communications functionality, input instructions for facilitating input functionality, output instructions for facilitating output functionality, and location instruction for facilitating location functionality, and instructions for operating the user client 31.

The computer readable mediums comprises instructions, executable by the processor(s) to implement and perform the features, functions, operations, and processes described herein in reference to the user client 31, the second user client 31b, and/or to other client side functionality.

The operating system can perform tasks, such as managing files and directories on the computer readable mediums, managing traffic on the one or more communication channels 192, recognizing input from input devices of the I/O system 198, and providing output to output devices of the I/O system, among other tasks. The network communications instructions can enable the establishing and maintain of network communications.

In some embodiments, the user client architecture for use with housings 220, 230 comprises the processor 194, the memory 195, the I/O system 198, and the communication system 200. The memory can comprise the instructions executable by the processor to carry out the operations and processes of the user client of housings 220, 230. The I/O system comprises input components comprising the buttons 224, 238, 240, 242. The I/O system comprises output components comprising the illuminating elements 222, 232, 243, 236. The communications system 202 is configured to send and receive information to and from other devices or systems, such as the cognitive authority.

In some embodiments, the housing 217, 220, 230 comprise other inputs, outputs, and functionality. For example the user client can be implemented on a key fob or an ID fob. The key fob may be used for accessing a vehicle, building, a location, or a secure area. The key fob may comprise a radio frequency transmitter/transceiver using radio frequency, or other wireless communication protocols, to communicate with an electronic control device of the vehicle, building, a location, or a secure area for requesting and receiving access thereto and/or operation thereof.

In some embodiments, the user client architecture comprises processing circuitry. The processing circuitry may comprise one or more of microprocessor(s), microcontroller(s), a hardware circuit(s), application-specific integrated circuit(s) (ASIC), digital signal processor(s) (DSP), field-programmable gate array(s) (FPGA), discrete logic circuit(s), or combinations thereof for performing the operations of the user client 31, 31b or other client side functionality. The processing circuitry may be connected, as shown for the processor(s) 194 in FIG. 26, to the other components, including memory 196. In some embodiments, instead of or in addition to storing instructions on the memory 196 as described above, instructions may be implemented by the processing circuitry.

In some embodiments, the computing architecture for the target computing resource 32 comprises processing circuitry. The processing circuitry may comprise one or more of microprocessor(s), microcontroller(s), a hardware circuit(s), application-specific integrated circuit(s) (ASIC), digital signal processor(s) (DSP), field-programmable gate array(s) (FPGA), discrete logic circuit(s), or combinations thereof for performing the operations of the target computing resource 32. The processing circuitry may be connected, as shown for the processor(s) 174 in FIG. 25, to the other components, including memory 178. In some embodiments, instead of or in addition to storing instructions on the memory 178 as described above, instructions may be implemented by the processing circuitry.

The steps, functions, processes, operations, and capabilities described herein can be provided in the form of instructions stored in a non-transitory computer readable medium and executable by a processor of a computing device to achieve the corresponding functions, processes, operations, capabilities, or results.

From the foregoing, it will be observed that numerous variations and modifications may be affected without departing from the spirit and scope of the invention. It is to be understood that no limitation with respect to the specific apparatus illustrated herein is intended or should be inferred. For example, one or more component embodiments may be combined, modified, removed, or supplemented to form further embodiments within the scope of the invention. Further, steps could be added or removed from the processes described. Therefore, other embodiments and implementations are within the scope of the invention.

1. A computer-implemented method of controlling access to a computing resource, comprising the steps of:
receiving a request for access to a computing resource, where access to the computing resource is restricted;
administering a cognitive readiness test on a client electronic device, where the cognitive readiness test comprises a presentation of a stimulus on the client electronic device;
controlling access to the computing resource based on a reaction time of a user to the stimulus of the cognitive readiness test.

2. The method of claim 1, comprising the step of:
determining the reaction time of the user to the stimulus of the cognitive readiness test is within a predefined range of acceptable reaction times; and,
wherein the step of controlling access comprises the step of granting access to the computing resource in response to determining the reaction time of the user to the stimulus of the cognitive readiness test is within the predefined range of acceptable reaction times.

3. The method of claim 2, wherein the step of controlling access comprises the step of continuing previously granted access to the computing resource.

4. The method of claim 2, comprising the steps of:
receiving a one or more authentication credentials of the user from the client electronic device;
determining the one or more authentication credentials are valid; and,
wherein the step of granting access is further defined in that access is granted to the computing resource in response to determining the one or more authentication credentials are valid and determining the reaction time of the user to the cognitive readiness test is within the predefined range of acceptable reaction times.

5. The method of claim 4, comprising the step of redirecting the user to an authentication authority after receiving the request for access to the computing resource, and wherein the step of receiving the one or more authentication credentials comprises receiving the one or more authentication credentials at the authentication authority.

6. The method of claim 1, wherein the step of administering comprises the step of presenting the cognitive readiness test on a graphical user interface of the client electronic device.

7. The method of claim 6, comprising the step of measuring, with the cognitive readiness test, a cognitive marker indicative of a user's cognitive readiness.

8. The method of claim 6, comprising the step of measuring the reaction time of the user on the client electronic device to the stimulus presented in the cognitive readiness test by measuring a time between the presentation of the stimulus on the graphical user interface and a receipt of a predefined user input on the client electronic device.

9. The method of claim 8, wherein the step of measuring the reaction time comprises measuring the reaction time on the client electronic device and sending the reaction time to a cognitive authority.

10. The method of claim 8, wherein the step of administering is further defined in that the stimulus is a bar intersecting with a goal element.

11. The method of claim 10, wherein the step of administering is further defined in that the bar moves along a circular track toward the goal element.

12. The method of claim 8, wherein the step of administering is further defined in that the stimulus is a block arriving in a goal area between a start element and an end element.

13. The method of claim 8, wherein the step of administering is further defined in that the stimulus is a question.

14. The method of claim 8, wherein the step of administering is further defined in that the stimulus is an illumination or a color change of a user interface element on the graphical user interface.

15. The method of claim 5, wherein the step of administering comprises the step of instructing the client electronic device to prompt the user to take the cognitive readiness test, and the step of receiving from the user one or more authentication credentials at the authentication authority is further defined in that the authentication authority is a single sign-on authority.

16. The method of claim 1, wherein the step of administering comprises the step of sending the cognitive readiness test to the client electronic device.

17. The method of claim 1, wherein the step of receiving and the step of controlling are further defined in that the computing resource is selected from the group consisting of: a software, a computer, a computer system, a system resource, a website, and a webpage.

18. The method of claim 2, wherein the step of granting access is further defined in that the predefined range of acceptable reaction times is a user personalized predefined range of acceptable reaction times determined based on one or more previous reaction times of one or more prior cognitive readiness tests taken by the user.

19. The method of claim 18, wherein the user personalized predefined range of acceptable reaction times is further defined as within an acceptable deviation range from an average of two or more previous reaction times of two or more prior cognitive readiness tests taken by the user.

20. The method of claim 4, wherein the step of receiving the one or more authentication credentials comprises the step of receiving a one-time use password or a second input after receiving a first set of the one or more authentication credentials.

21. The method of claim 20, comprising the step of, before receiving the one-time use password or the second input, sending a sent one-time use password or a request for the second input to the client electronic device.

22. (canceled)

23. The method of claim 1, comprising the steps of:
determining the reaction time of the user to the stimulus of the cognitive readiness test is not within a predefined range of acceptable reaction times; and,
wherein the step of controlling access is a step of denying access to the computing resource in response to determining the reaction time of the user to the cognitive readiness test is not within the predefined range of acceptable reaction times.

24. The method of claim 23, the method further defined in that the user is a first user, and the method comprising the step of:
notifying a second user in response to determining the reaction time of the first user to the cognitive readiness test is not within the predefined range of acceptable reaction times.

25. The method of claim 23, comprising the step of:
recording in a data store the reaction time of the user in response to determining the reaction time of the user to the cognitive readiness test is not within the predefined range of acceptable reaction times.

26. A computer program product on a non-transitory computer readable medium having a plurality of program instructions stored thereon, which when executed by a processor, causes the processor to perform operations comprising:
instructing the administration of a cognitive readiness test on a client electronic device, where the cognitive readiness test comprises a presentation of a stimulus on the client electronic device; and,
controlling access to a computing resource based on a reaction time of a user to the stimulus of the cognitive readiness test administered on the client electronic device.

27. (canceled)

28. A computing resource access control system, comprising:
a processor,
a memory comprising a plurality of program instructions stored thereon that are executable by the processor to perform operations comprising,
instructing an administration of a cognitive readiness test on a client electronic device of a user in response to a request for access to a computing resource, where the cognitive readiness test comprises a presentation of a stimulus on the client electronic device; and,
controlling access to the computing resource based on a reaction time of the user to the stimulus of the cognitive readiness test.

29. The system of claim 28, wherein the plurality of program instructions comprise program instructions executable by the processor to perform operations comprising determining the reaction time of the user to the stimulus of the cognitive readiness test is within a predefined range of acceptable reaction times; and wherein the controlling access is further defined as granting access to the computing resource in response to determining the reaction time of the user to the cognitive readiness test is within the predefined range of acceptable reaction times.

30. The system of claim 28, wherein the controlling access comprises continuing previously granted access to the computing resource.

31. The system of claim 29, wherein the plurality of program instructions comprise program instructions executable by the processor to perform operations comprising:
receiving a one or more authentication credentials of the user from the client electronic device;
determining the one or more authentication credentials are valid; and,
wherein controlling access is further defined in that access is granted to the computing resource in response to determining the one or more authentication credentials are valid and determining the reaction time of the user is within the predefined range of acceptable reaction times.

32. The system of claim 31, wherein the plurality of program instructions comprise program instructions executable by the processor to perform operations comprising redirecting the user to an authentication authority after receiving a request for access to the computing resource, and wherein receiving the one or more authentication credentials comprises receiving the one or more authentication credentials at the authentication authority.

33. The system of claim 28, comprising
a client electronic device having
a client processor and a graphical user interface in communication with the processor,
a client memory comprising a plurality of client program instructions stored thereon that are executable by the processor to perform operations comprising:
administering the cognitive readiness test on the graphical user interface.

34. The system of claim 33, wherein the plurality of client program instructions comprise client program instructions executable by the client processor to perform operations comprising measuring, with the cognitive readiness test, a cognitive marker indicative of a user's cognitive readiness.

35. The system of claim 34, wherein the measuring the reaction time of the user on the client electronic device to the stimulus presented in the cognitive readiness test comprises measuring a time between the presentation of the stimulus on the graphical user interface and a receipt of a predefined user input on the client electronic device.

36. The system of claim 34, wherein the step of administering comprises the step of instructing the client electronic device to prompt the user to take the cognitive readiness test.

37.-129. (canceled)
